(19) United States
(12) Patent Application Publication
(10) Pub. No.: US 2009/0131705 A1
(43) Pub. Date: May 21, 2009

(54) Practical, Cost-Effective Synthesis of Ubiquinones

(75) Inventors: Bruce H. Lipshutz, Goleta, CA (US); Volker Berl, Strasbourg (FR); Karin Schein, Ludwigshafen (DE); Frank Wetterich, Wachenheim (DE)

Correspondence Address: MORGAN, LEWIS & BOCKIUS LLP (SF), One Market, Spear Street Tower, Suite 2800, San Francisco, CA, 94105, US

(73) Assignee: Zymes, LLC

(21) Appl. No.: 12/096,372

(22) PCT Filed: Dec. 12, 2006
(86) PCT No.: PCT/US06/47257

§ 371 Date: Nov. 4, 2008

(51) Int. Cl. C07F 5/06; C07C 49/403

(52) U.S. Cl. 556/170; 568/337

(57) ABSTRACT

The present invention provides a convergent method for the synthesis of ubiquinones and ubiquinone analogues. Also provided are precursors of ubiquinones and their analogues that are useful in the methods of the invention. The invention further provides an improved method for the carboalumination of alkyne substrates.

### CROSS-REFERENCE TO RELATED APPLICATIONS

This application is a U.S. National Phase of PCT Patent Application No. PCT/US2006/047257 filed Dec. 12, 2006 and claims priority to U.S. patent application Ser. No. 11/304,023 filed Dec. 15, 2005, the disclosures of which are incorporated herein by reference in their entirety for all purposes.

### BACKGROUND OF THE INVENTION

The ubiquinones, also commonly called coenzyme Qₙ (n=1-12), constitute essential cellular components of many life forms. In humans, CoQ₁₀ is the predominant member of this class of polyprenoidal natural products and is well-known to function primarily as a redox carrier in the respiratory chain (Lenaz, COENZYME Q. BIOCHEMISTRY, BIOENERGETICS, AND CLINICAL APPLICATIONS OF UBIQUINONE, Wiley-Interscience: New York (1985); Trumpower, FUNCTION OF UBIQUINONES IN ENERGY CONSERVING SYSTEMS, Academic Press, New York (1982); Thomson, R. H., NATURALLY OCCURRING QUINONES, 3rd ed., Academic Press, New York (1987); Bliznakov et al., THE MIRACLE NUTRIENT COENZYME Q₁₀, Bantom Books, New York (1987)).

Coenzyme Q plays an essential role in the orchestration of electron-transfer processes necessary for respiration. Almost all vertebrates rely on one or more forms of this series of compounds that are found in the mitochondria of every cell (i.e., they are ubiquitous, hence the alternative name “ubiquinones”). Although usually occurring with up to 12 prenoidal units attached to a p-quinone headgroup, CoQ₁₀ is the compound used by humans as a redox carrier. Oftentimes unappreciated is the fact that when less than normal levels are present, the body must construct its CoQ₁₀ from lower forms obtained through the diet, and that at some point in everyone's life span the efficiency of that machinery begins to drop. (Blizakov et al, supra) The consequences of this in vivo deterioration can be substantial; levels of CoQ₁₀ have been correlated with increased sensitivity to infection (i.e., a weakening of the immune system), strength of heart muscle, and metabolic rates tied to energy levels and vigor. In the United States, however, it is considered a dietary supplement, sold typically in health food stores or through mail order houses at reasonable prices. It is indeed fortunate that quantities of CoQ₁₀ are available via well-established fermentation and extraction processes (e.g., Sasikala et al., Adv. Appl. Microbiol., 41:173 (1995); U.S. Pat. Nos. 4,447,362; 3,313,831; and 3,313,826) an apparently more cost-efficient route relative to total synthesis. However, for producing lower forms of CoQ, such processes are either far less efficient or are unknown. Thus, the costs of these materials for research purposes are astonishingly high, e.g., CoQ₆ is ˜$22,000/g, and CoQ₉ is over $40,000/g. (Sigma-Aldrich Catalog, Sigma-Aldrich: St. Louis, pp. 306-307 (1998)).

Several approaches to synthesizing the ubiquinones have been developed over the past 3-4 decades, attesting to the importance of these compounds. Recent contributions have invoked such varied approaches as Lewis acid-induced prenoidal stannane additions to quinones, (Naruta, J. Org. Chem., 45:4097 (1980)) reiterative Pd(0)-catalyzed couplings of doubly activated prenoidal chains with allylic carbonates bearing the required aromatic nucleus in protected form (Eren et al., J. Am. Chem. Soc., 110:4356 (1988) and references therein), and a Diels-Alder, retro Diels-Alder route to arrive at the quinone oxidation state directly (Van Lient et al., Rec. Trav. Chim. Pays-Bays 113:153 (1994); and Rüttiman et al., Helv. Chim. Acta, 73:790 (1990)). Nonetheless, all are lengthy, linear rather than convergent, and/or inefficient. Moreover, problems in controlling double bond stereochemistry using, e.g., a copper(I)-catalyzed allylic Grignard-allylic halide coupling can lead to complicated mixtures of geometrical isomers that are difficult to separate given the hydrocarbon nature of the side chains (Yanagisawa, et al, Synthesis, 1130 (1991)).

Another method of producing ubiquinones has been developed by Negishi (Negishi, Org. Lett. 4(2): 261-264 (2002)). In this publication, Negishi describes a traditional carboalumination of unactivated alkynes. This method possesses some characteristics that limit its applicability for industrial uses. For example, the reactions in Negishi are conducted in chlorinated solvents, which can constitute a significant waste removal expense. In addition, the use of large amounts of ≧25 mole % of a zirconocene species in the carboalumination reaction creates vinylic alanes in the presence of zirconium salts that perform with less than optimal efficiency in subsequent coupling reactions with key chloromethylated quinones as substrates. Thus, the zirconecene salts necessitate their costly separation from the vinyl alane to be used in the coupling, significantly impacting the economic costs of the process.

For the reasons set forth above, a convergent method for the synthesis of the ubiquinones and their analogues which originates with a simple benzenoid precursor and proceeds with retention of the double bond stereochemistry would represent a significant advance in the synthesis of ubiquinones and their analogues. The present invention provides such a method and ubiquinone precursors of use in the method.

### SUMMARY OF THE INVENTION

The present invention provides an efficient and inexpensive method for preparing ubiquinones and structural analogues of these essential molecules. Also provided are new compounds that are structurally simple and provide a convenient, efficient and inexpensive entry into the method of the invention. In addition, new methods of carboalumination, such as the synthesis of vinyl alanes, are accomplished through the use of substituted and/or bridged metallocene catalysts.

Thus, in a first aspect, the present invention provides a compound according to Formula (I):

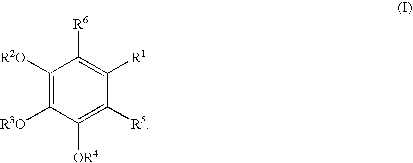

In Formula (I), R¹, R² and R³ are independently selected substituted or unsubstituted C₁-C₆ alkyl groups, e.g., methyl groups. R⁴ represents H, substituted or unsubstituted alkyl, e.g., methyl, or a protecting group. R⁵ is selected from branched, unsaturated alkyl, —CH(O) (formyl), and —CH₂Y, in which Y can be OR⁷, SR⁷, NR⁷R⁸, or a leaving group. R⁷ and R⁸ are independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. R⁶ is H, —OCH(O), or another group that is readily converted to a quinone carbonyl moiety.

In an exemplary embodiment, when R⁵ is —CH(O) or Y is a leaving group, e.g., halo, then R⁶ is OCH(O).

In a second aspect, the invention provides compounds according to Formula (II):

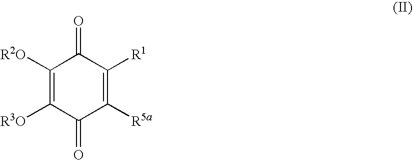

in which R¹, R² and R³ are as described for Formula (I) and R^{5a} is —CH(O) or CH₂OR⁷.

In a third aspect, the present invention provides methods for preparing a ubiquinone according to Formula (III):

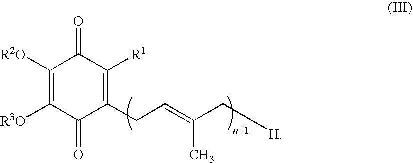

In Formula (III), each of R¹, R² and R³ are substituents as described for Formula (I), and the subscript n represents an integer from 0 to 19.

Thus, an exemplary method of the invention includes contacting a compound according to Formula (I):

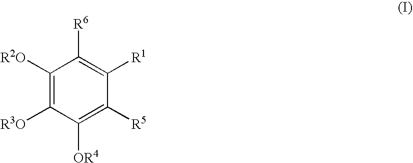

with a compound according to Formula (IV):

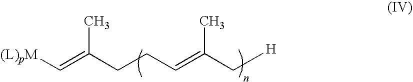

in which each L is an independently selected organic ligand or substituent, e.g., substituted or unsubstituted alkyl; M is aluminum; p is 1 or 2; and n is an integer from 0 to 19. Each of the organic ligands (substituents) L, can be the same or different. R¹-R⁶ are as discussed above.

The mixture of compounds according to Formulae (I) and (IV) are contacted with a coupling catalyst, e.g., Ni(0) that is effective at catalyzing coupling between a benzylic carbon atom, such as that in Formula (I), and an organometallic species according to Formula (IV). The coupling of the compounds of Formulae (I) and (IV) forms a compound according to Formula (V):

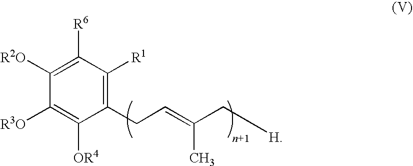

R⁴ is preferably removed from the compound according to Formula (V) to produce a compound according to Formula (VI), in which n represents an integer from 0 to 19:

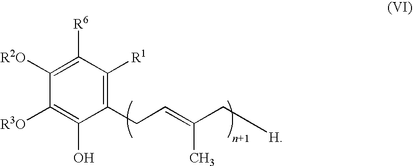

Contacting the compound according to Formula (VI) with an oxidant yields a compound according to Formula (III).

In another aspect, the invention provides a method for preparing a ubiquinone by direct coupling of an alkene to a substituted-methylene quinone (e.g., an ether, sulfonate, etc.). Thus, a compound according to Formula (II):

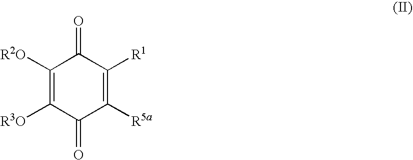

is contacted with a compound according to Formula (IV) in the presence of a coupling catalyst. An exemplary coupling catalyst is a nickel catalyst.

In a still further aspect, the invention provides a reaction pathway that includes the direct coupling of a compound according to Formula (IV) with a halomethyl quinone having the formula:

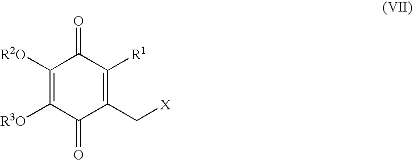

in which X is a leaving group, e.g., halogen, and R¹—R³ are as defined above.

In still another aspect the invention provides a method of carboaluminating an alkyne substrate, forming a species with an alkyl moiety bound to aluminum, said method comprising contacting said alkyne substrate with (L)ₚ₊₁M and x molar equivalents of water or R²⁰OH, or, when each L is methyl, with x molar equivalents of water, R²⁰OH or methylaluminoxane relative to said alkyne substrate, wherein
0≦x≦1;
each L is independently selected from substituted or unsubstituted alkyl, alkoxy, aryl or aryloxy with 1 to 10 carbon atoms;
M is aluminum;
p is 1 or 2 and,
R²⁰ is branched or unbranched alkyl with 1 to 15 carbon atoms, optionally substituted with 1 to 5 hydroxy substituents,
   thus carboaluminating said alkyne substrate.

The present invention also provides a method of preparing ubiquinones and their analogues that does not require the use of halogenated reaction solvents.

Also provided is a method of preparing a compound according to Formula (VII) as shown in FIG. 1. The invention also provides novel methods of purification that allow for ready access to a chloromethylated quinone (VII, X═Cl), prepared in two steps from trimethoxytoluene, as outlined in FIG. 4, that is suitable for use directly in the coupling step to produce CoQₙ₊₁.

Other methods of the invention utilize a metal catalyst, e.g., a zirconocene or titanocene, in a catalytic process to carboaluminate, e.g., carboaluminate a substrate. An exemplary compound formed by this method is set forth in Formula (IV).

In still a further aspect, the invention provides a mixture comprising:

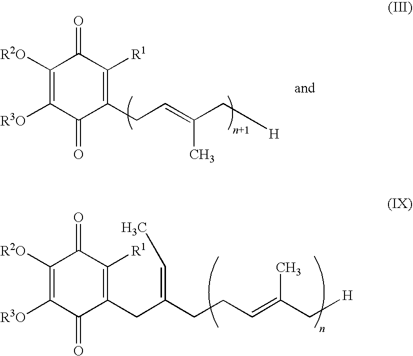

wherein R¹, R² and R³ are members independently selected from substituted or unsubstituted C₁-C₆ alkyl groups, and n is an integer from 0 to 19.

In another aspect, the invention provides a method for carboaluminating an alkyne substrate and forming a first vinyl alane. This method comprises contacting an alkyne substrate with (1) a carboalumination catalyst and (2) (L)ₚ₊₁Al in order to form the vinyl alane. The carboalumination catalyst can be any substituted and/or bridged metallocene that is capable of performing the method of the invention. In an exemplary embodiment, the carboalumination catalyst has a structure according to the following formula:

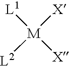

wherein M is a member selected from zirconium, titanium and hafnium. L¹ and L² are members independently selected from substituted or unsubstituted aryl, substituted or unsubstituted heteroaryl and substituted or unsubstituted cycloalkyl. L¹ and L² can optionally be covalently connected through a bridging ligand, Z. This method also has the proviso that L¹ and L² cannot both be unsubstituted cyclopentadienyl and L¹ and L² are not covalently connected to form substituted or unsubstituted tetraphenylporphyrinyl. X′ and X″ are members independently selected from hydrogen, substituted or unsubstituted alkyl, a leaving group and an empty coordination site on M. The p in (L)ₚ₊₁Al is an integer between 1 and 2. Each L in (L)ₚ₊₁Al is a member independently selected from substituted or unsubstituted alkyl, substituted or unsubstituted alkoxy, substituted or unsubstituted aryl and substituted or unsubstituted aryloxy, thus forming said first vinyl alane

In another aspect, the invention provides a method for the preparation of an alkene which comprises contacting a vinyl alane having a structure according to Formula (XL) with a compound having a structure according to Formula (XLIX)

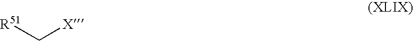

wherein X′″ is a leaving group, in the presence of a coupling catalyst effective at catalyzing coupling between the methylene carbon in Formula (XLIX) and C* in Formula (XL), thereby preparing the alkene. In this invention, the alkene has a structure according to Formula (XLVIII):

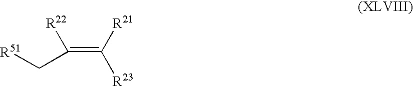

wherein R²¹ is member selected from substituted or unsubstituted alkyl, substituted or unsubstituted cycloalkyl, substituted or unsubstituted heteroalkyl, substituted or unsubstituted heterocycloalkyl, substituted or unsubstituted aryl, and substituted or unsubstituted heteroaryl. R²² is a member selected from hydrogen, substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, OR²⁶, NR²⁶R²⁷ and —SiR²⁶R²⁷R²⁸. R²³ is a member selected from substituted or unsubstituted alkyl. R⁵¹ is a member selected from substituted or unsubstituted C₂ to C₂₀ alkyl, substituted or unsubstituted C₂ to C₂₀ cycloalkyl, substituted or unsubstituted C₂ to C₂₀ heteroalkyl, substituted or unsubstituted C₂ to C₂₀ heterocycloalkyl, substituted or unsubstituted C₃ to C₂₀ aryl and substituted or unsubstituted C₃ to C₂₀ heteroaryl.

In another aspect, the invention provides a method of using a carboalumination catalyst to form a vinyl alane, said method comprising contacting a carboalumination catalyst with an alkyne substrate, thereby forming said vinyl alane. In this aspect, the carboalumination catalyst has a structure which is a member according to Formula (XLIII) or Formula (XLIV):

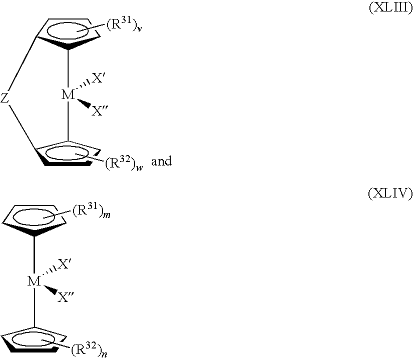

wherein M is a member selected from zirconium, titanium and hafnium. X′ and X″ are members independently selected from hydrogen and a leaving group, wherein said leaving group is a member selected from halogen, OR³⁶, OC(O)R³⁶, OS(O)R³⁶, OSO₂R³⁶, SR³⁶, S⁺(R³⁶)₂, OP(O)R³⁶R³⁷, P(O)N(R³⁶)₂(R³⁶)₂, P⁺(R³⁶)₃, P(O)R³⁸R³⁶R³⁹R³⁶. R³⁶ and R³⁷ are members independently selected from substituted or unsubstituted alkyl, substituted or unsubstituted aryl, substituted or unsubstituted heteroaryl and substituted or unsubstituted heterocycloalkyl and R³⁸ and R³⁹ are members independently selected from S and O. The indices v and w are integers independently selected from 0 to 4. When v is greater than 1, then two or more of R³¹, taken together with the carbons to which they are attached, optionally form a ring. When w is greater than 1, then two or more R³², taken together with the carbons to which they are attached, optionally form a ring. The index m is an integer selected from 0 to 5 and the index n is an integer selected from 0 to 5, with the proviso that at least one of m and n is not 0. When m is greater than 1, then two or more of R³¹, taken together with the carbons to which they are attached, optionally form a ring and when n is greater than 1, then two or more of R³², taken together with the carbons to which they are attached, optionally form a ring. R³¹ and R³² are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, OR²⁶, NR²⁶R²⁷ and —SiR²⁶R²⁷R²⁸. R²⁶, R²⁷ and R²⁸ are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl and substituted or unsubstituted heteroaryl. Z is a member selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl and —SiR^{29a}R^{29b}. R^{29a} and R^{29b} are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, —OR^{26a}, —SR^{26a}, —NR^{26a}R^{26b} and —PR^{26a}R^{26b}. R^{26a} and R^{26b} are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl and substituted or unsubstituted heteroaryl.

Other objects and advantages of the invention will be apparent to those of skill in the art from the detailed description that follows.

### BRIEF DESCRIPTION OF THE DRAWINGS

FIG. 1 sets forth representative intermediates and transformations of use in the process of the invention.

FIG. 2 sets forth a method of producing an ubiquinone.

FIG. 3 sets forth another method of producing an ubiquinone.

FIG. 4 sets forth a method of converting an aromatic moiety into a substituted methylene quinone and a haloquinone.

### DETAILED DESCRIPTION OF THE INVENTION AND THE PREFERRED EMBODIMENTS

### Definitions

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multi-valent radicals, having the number of carbon atoms designated (i.e. C₁-C₁₀ means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)ethyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below as “heteroalkyl,” “cycloalkyl” and “alkylene.” The term “alkylene” by itself or as part of another substituent means a divalent radical derived from an alkane, as exemplified by —CH₂CH₂CH₂CH₂—. Typically, an alkyl group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The terms “alkoxy,” “alkylamino” and “alkylthio” refer to those groups having an alkyl group attached to the remainder of the molecule through an oxygen, nitrogen or sulfur atom, respectively. Similarly, the term “dialkylamino” is used in a conventional sense to refer to —NR′R″ wherein the R groups can be the same or different alkyl groups.

The term “acyl” or “alkanoyl” by itself or in combination with another term, means, unless otherwise stated, a stable straight or branched chain, or cyclic hydrocarbon radical, or combinations thereof, consisting of the stated number of carbon atoms and an acyl radical on at least one terminus of the alkane radical.

The term “heteroalkyl,” by itself or in combination with another term, means, unless otherwise stated, a stable straight or branched chain, or cyclic hydrocarbon radical, or combinations thereof, consisting of the stated number of carbon atoms and from one to three heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —Si(Me)₂-, —Si(CH(Me)₂)₂—, —Si(R²⁸C(Me)₃)—, —Si(Ph)₂-, —CH₂OSi(Me₂)OCH₂— and —CH═CH—N(CH₃)—CH₃. Up to two heteroatoms may be consecutive, such as, for example, —CH₂—NH—OCH₃ and —CH₂—O—Si(CH₃)₃. Also included in the term “heteroalkyl” are those radicals described in more detail below as “heteroalkylene” and “heterocycloalkyl.” The term “heteroalkylene” by itself or as part of another substituent means a divalent radical derived from heteroalkyl, as exemplified by —CH₂—CH₂—S—CH₂CH₂— and —CH₂—S—CH₂—CH₂—NH—CH₂—. For heteroalkylene groups, heteroatoms can also occupy either or both of the chain termini. Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include cyclopentyl, cyclohexyl, 1-cyclohexenyl, 3-cyclohexenyl, cycloheptyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “fluoroalkyl,” are meant to include monofluoroalkyl and polyfluoroalkyl.

The term “aryl,” employed alone or in combination with other terms (e.g., aryloxy, arylthioxy, arylalkyl) means, unless otherwise stated, an aromatic substituent which can be a single ring or multiple rings (up to three rings), which are fused together or linked covalently. “Heteroaryl” are those aryl groups having at least one heteroatom ring member. Typically, the rings each contain from zero to four heteroatoms selected from N, O, and S, wherein the nitrogen and sulfur atoms are optionally oxidized, and the nitrogen atom(s) are optionally quaternized. The “heteroaryl” groups can be attached to the remainder of the molecule through a heteroatom. Non-limiting examples of aryl and heteroaryl groups include phenyl, cyclopentadienyl anion, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl ring systems are selected from the group of acceptable substituents described below. The term “arylalkyl” is meant to include those radicals in which an aryl group is attached to an alkyl group (e.g., benzyl, phenethyl, pyridylmethyl and the like) or a heteroalkyl group (e.g., phenoxymethyl, 2-pyridyloxymethyl, 3-(1-naphthyloxy)propyl, and the like).

Each of the above terms (e.g., “alkyl,” “heteroalkyl” and “aryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from, for example: hydrogen, -halogen, —R³³, —OH, —OR³³, —SH, —SR³³, —NH₂, —NO₂, —NR³³R³⁴, ═NR³³, ═N—OR³³, —CN, —N₃, —PR³³R³⁴, —SiR³³R³⁴R³⁵, —OSiR³³R³⁴R³⁵, —C(O)OR³³, ═O, —OC(O)R³³, —C(O)R³³, —CO₂R³³, CONR³³R³⁴, —OC(O)NR³³R³⁴, —NR³³C(O)R³⁴, —NR³³—C(O)NR³⁴R³⁵, —NR³³C(O)₂R³⁴, —NH—C(NH₂)═NH, —NR′C(NH₂)═NH, —NH—C(NH₂)═NR³³, —S(O)R³³, —S(O)₂R³³, and —S(O)₂NR³³R³⁴ in a number ranging from zero to (2N+1), where N is the total number of carbon atoms in such radical. R³³, R³⁴ and R³⁵ each independently refer to hydrogen, substituted and unsubstituted alkyl, substituted and unsubstituted heteroalkyl, substituted and unsubstituted cycloalkyl, substituted and unsubstituted heterocycloalkyl, substituted and unsubstituted aryl, substituted and unsubstituted heteroaryl, aryl substituted with 1-3 halogens, unsubstituted alkyl, alkoxy or thioalkoxy groups, or aryl-(C₁-C₄)alkyl groups. When R³³ and R³⁴ are attached to the same nitrogen atom, they can be combined with the nitrogen atom to form a 5-, 6-, or 7-membered ring. For example, —NR³³R³⁴ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl and heteroaryl groups are varied and are selected from: hydrogen, -halogen, —R³³, —OH, —OR³³, —SH, —SR³³, —NH₂, —NO₂, —NR³³R³⁴, ═NR³³, ═N—OR³³, —CN, —N₃, —PR³³R³⁴, —SiR³³R³⁴R³⁵, —OSiR³³R³³R³⁵, —C(O)OR³³, ═O, —OC(O)R³³, C(O)R³³, —CO₂R³³, CONR³³R³⁴, —OC(O)NR³³R³⁴, —NR³³—C(O)R³⁴, —NR³³—C(O)NR³⁴R³⁵, —NR³³C(O)₂R³⁴, —NH—C(NH₂)═NH, —NR′C(NH₂)═NH, —NH—C(NH₂)═NR³³, —S(O)R³³, —S(O)₂R³³, and —S(O)₂NR³³R³⁴, —CH(Ph)₂, perfluoro(C₁-C₄)alkoxy, and perfluoro(C₁-C₄)alkyl, in a number ranging from zero to the total number of open valences on the aromatic ring system; and where R³³, R³⁴ and R³⁵ each independently refer to hydrogen, substituted and unsubstituted alkyl, substituted and unsubstituted heteroalkyl, substituted and unsubstituted cycloalkyl, substituted and unsubstituted heterocycloalkyl, substituted and unsubstituted aryl, substituted and unsubstituted heteroaryl.

Two of the substituents on adjacent atoms of the aryl ring may optionally be replaced with a substituent of the formula -T-C(O)—(CH₂)_{q}—U—, wherein T and U are independently —NH—, —O—, —CH₂— or a single bond, and the subscript q is an integer of from 0 to 2. Alternatively, two of the substituents on adjacent atoms of the aryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR³³, or a single bond, and r is an integer of from 1 to 3. One of the single bonds of the new ring so formed may optionally be replaced with a double bond. Alternatively, two of the substituents on adjacent atoms of the aryl ring may optionally be replaced with a substituent of the formula —(CH₂)ₛ—X—(CH₂)ₜ—, where s and t are independently integers of from 0 to 3, and X is —O—, —NR³³—, —S—, —S(O)—, —S(O)₂—, or —S(O)₂NR³³—. The substituent R³³ in —NR³³— and —S(O)₂NR³³— is selected from hydrogen or unsubstituted (C₁-C₆)alkyl.

As used herein, the term “heteroatom” is meant to include, for example, oxygen (O), nitrogen (N), sulfur (S) and silicon (Si).

Certain compounds of the present invention possess asymmetric carbon atoms (optical centers) or double bonds; the racemates, diastereomers, geometric isomers and individual isomers are all encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein, the term “leaving group” refers to a portion of a substrate that is cleaved from the substrate in a reaction. The leaving group is an atom (or a group of atoms) that is displaced as stable species taking with it the bonding electrons. Typically the leaving group is an anion (e.g., Cl⁻) or a neutral molecule (e.g., H₂O). Exemplary leaving groups include a halogen, OC(O)R³⁶, OP(O)R³⁶R³⁷, OS(O)R³⁶, and OSO₂R³⁶. R³⁶ and R³⁷ are members independently selected from substituted or unsubstituted alkyl, substituted or unsubstituted aryl, substituted or unsubstituted heteroaryl and substituted or unsubstituted heterocycloalkyl. Useful leaving groups include, but are not limited to, other halides, sulfonic esters, oxonium ions, alkyl perchlorates, sulfonates, e.g., arylsulfonates, ammonioalkanesulfonate esters, and alkylfluorosulfonates, phosphates, carboxylic acid esters, carbonates, ethers, and fluorinated compounds (e.g., triflates, nonaflates, tresylates), S R³⁶, (R³⁶)₃P⁺, (R³⁶)₂S⁺, P(O)N(R³⁶)₂(R³⁶)₂, P(O)R³⁸R³⁶R³⁶ in which each R³⁶ is independently selected from the members provided in this paragraph and R³⁸ and R³⁹ are each either S or O. The choice of these and other leaving groups appropriate for a particular set of reaction conditions is within the abilities of those of skill in the art (see, for example, March J, ADVANCED ORGANIC CHEMISTRY, 2nd Edition, John Wiley and Sons, 1992; Sandler S R, Karo W, ORGANIC FUNCTIONAL GROUP PREPARATIONS, 2nd Edition, Academic Press, Inc., 1983; and Wade L G, COMPENDIUM OF ORGANIC SYNTHETIC METHODS, John Wiley and Sons, 1980).

“Protecting group,” as used herein refers to a portion of a substrate that is substantially stable under a particular reaction condition, but which is cleaved from the substrate under a different reaction condition. A protecting group can also be selected such that it participates in the direct oxidation of the aromatic ring component of the compounds of the invention. For examples of useful protecting groups, see, for example, Greene et al., PROTECTIVE GROUPS IN ORGANIC SYNTHESIS, 3rd ed., John Wiley & Sons, New York, 1999.

“Adsorbent”, as used herein refers to a material with the property to hold molecules of fluids without causing a chemical or physical change. Examples are Silica gel, Alumina, Charcoal, Ion exchange resins and others, characterized by high surface/volume ratio.

“Metallocene”, as used herein, refers to a compound consisting of one or more aromatic organic ligands bound to a metal.

### Introduction

The present invention provides an efficient and cost-effective route to the ubiquinones and their analogues. The present method is quite general and employs substituted and/or bridged metallocene catalysts to afford CoQₙ₊₁ and analogues as well as systems found in vitamins K₁ and K₂ and their analogues. The invention also provides compounds that are useful in the method of the invention.

As set forth herein, the invention also provides useful improvements in methods of purifying substituted-methylene quinones from halo-quinones, and methods of improved efficiency for carboaluminating an alkyne substrate.

### The Compounds

In a first aspect, the present invention provides a compound according to Formula (I):

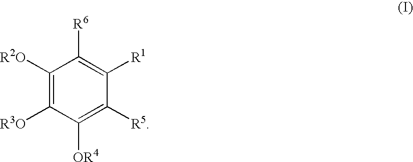

In Formula (I), R¹, R² and R³ are independently selected substituted or unsubstituted C₁-C₆ alkyl groups, preferably methyl groups. R⁴ represents H, substituted or unsubstituted alkyl, preferably methyl, a metal ion or a protecting group. R⁵ can be selected from branched, unsaturated alkyl, —CH(O), and —CH₂Y, in which Y is OR⁷, SR⁷, NR⁷R⁸, or a leaving group. In an exemplary embodiment, Y is OR^{7a}, in which R^{7a}, together with the oxygen to which it is bound, forms a leaving group.

R⁷ and R⁸ can be independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. R⁶ is H, OH or —OCH(O), or another group that is readily converted to a quinone ketone moiety or a phenyl H atom.

Exemplary substituents R^{7a} include —SOR⁹, —SO₂R⁹, —C(O)R⁹, —C(O)OR⁹, —P(O)OR⁹OR¹⁰, —P(O)N(R⁹)₂(R¹⁰)₂, and —P(O)R⁹R¹⁰. R⁹ and R¹⁰ can be members independently selected from substituted or unsubstituted alkyl, substituted or unsubstituted aryl, substituted or unsubstituted heteroaryl and substituted or unsubstituted heterocycloalkyl.

In an exemplary embodiment, when R⁵ is —CH(O) or Y is a leaving group, e.g., halo, then R⁶ is —OCH(O). In another exemplary embodiment, R⁵ has a structure according to Formula (VIII):

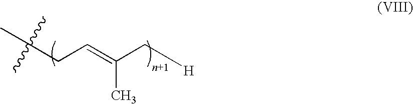

in which the symbol n can be selected from the integers from 0 to 19. In an exemplary embodiment, the symbol n can be selected from the integers from 0 to 13. In another exemplary embodiment, the symbol n can be selected from the integers from 4 to 10.

In a second aspect, the invention provides compounds according to Formula (II):

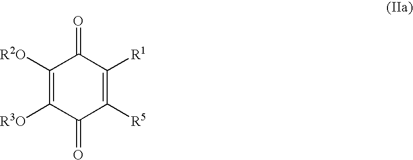

in which R¹, R² and R³, and R⁵ are as described for Formula (I). In another exemplary embodiment R⁵ has a structure according to Formula (VIII):

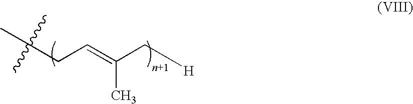

in which the symbol n can be selected from the integers from 0 to 19. In an exemplary embodiment, the symbol n can be selected from the integers from 0 to 13. In another exemplary embodiment, the symbol n can be selected from the integers from 4 to 10.

Exemplary compounds of the invention according to Formulae I and II include:

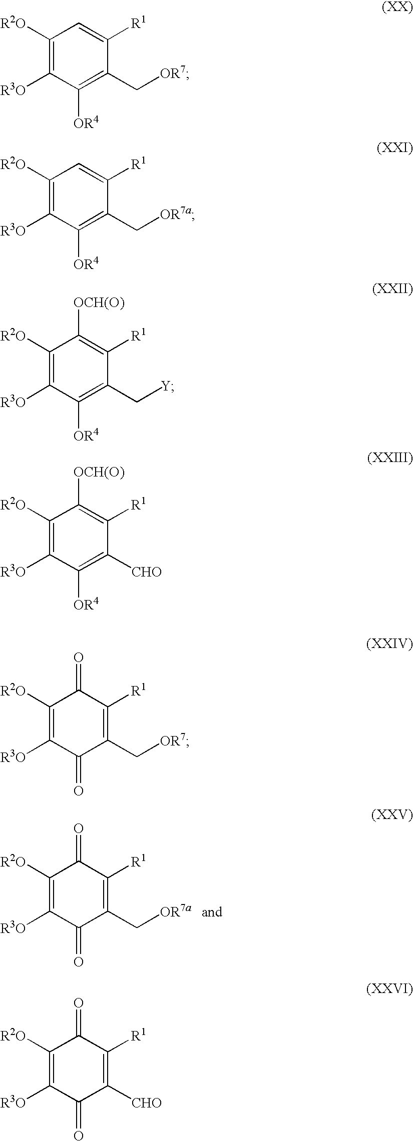

in which the identity of the substituents is as discussed hereinabove.

In still further exemplary compounds according to the invention, R¹, R², and R³ can be methyl; and R⁴ is methyl or H. In another exemplary embodiment, R^{7a} can be SOR⁹, SO₂R⁹, C(O)R⁹, C(O)OR⁹, P(O)OR⁹OR¹⁰, P(O)N(R⁹)₂(R¹⁰)₂, and P(O)R⁹R¹⁰. R⁹ and R¹⁰ can be independently selected from substituted or unsubstituted alkyl, substituted or unsubstituted aryl, substituted or unsubstituted heteroaryl and substituted or unsubstituted heterocycloalkyl.

Further exemplary compounds of the invention include:

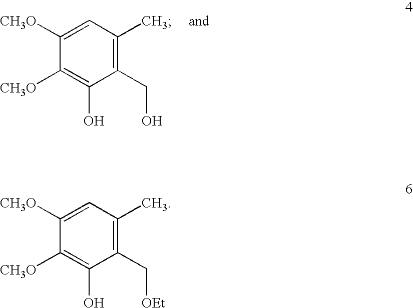

The invention also provides a mixture comprising the regioisomers according to Formulae (III) and (IX):

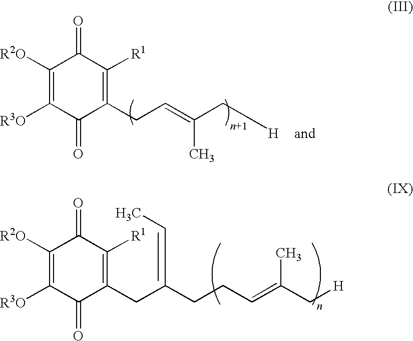

in which the symbols R¹, R² and R³ independently represent substituted or unsubstituted C₁-C₆ alkyl groups; and the symbol n is an integer from 0 to 19. In a preferred embodiment R¹, R² and R³ in Formulae (III) and (IX) is methyl. Further preference is given to mixtures of compounds of Formulae (III) and (IX) in which the molar ratio of the compound of Formula (III) to the compound of Formula (IX) is at least 8:1.

### Synthesis of the Compounds and Methods of the Invention

Techniques useful in synthesizing the compounds of the invention are both readily apparent and accessible to those of skill in the relevant art. The discussion below is offered to illustrate certain of the diverse methods available for use in assembling the compounds of the invention, it is not intended to define the scope of reactions or reaction sequences that are useful in preparing the compounds of the present invention.

### Synthesis of the Starting Materials

### Synthesis of the Substituted Methylene Moiety

The substituted methylene moieties of the invention are prepared by art-recognized methods or modifications thereof. For example, the synthesis of quinones functionalized with a halomethyl group can be accomplished using methods such as that described by Lipshutz (Lipshutz et al., J. Am. Chem. Soc. 121: 11664-11673 (1999)), the disclosure of which is incorporated herein by reference. In addition, the synthesis of substituted methylene aromatic moieties, such as phenols, can be accomplished using methods described by U.S. Pat. No. 6,545,184 to Lipshutz et al., the disclosure of which is also herein incorporated by reference.

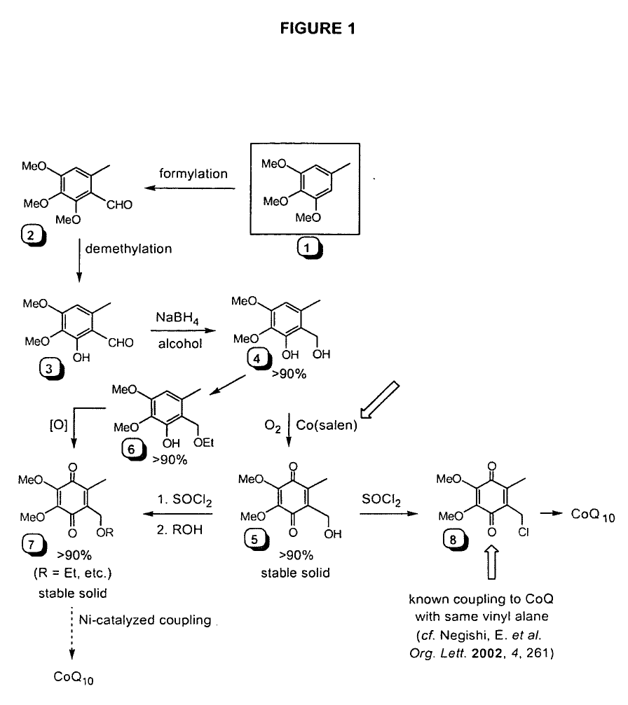

In one aspect, the invention provides a method of preparing a substituted methylene moiety present in quinone (XXVIII) by performing the following transformation:

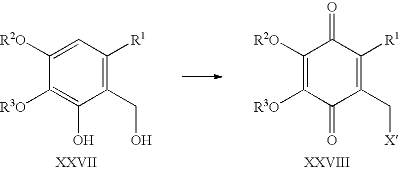

in which R¹, R² and R³ can each be independently selected from substituted or unsubstituted C₁-C₆ alkyl groups. X′ is OH or a leaving group. In an exemplary embodiment, R¹, R² and R³ are methyl. In another exemplary embodiment, the method further comprises the synthesis of the substituted methylene moiety. Representative transformations for preparing this and other selected compounds of the invention are displayed in FIG. 1. Commercially available 1 is formulated, yielding aldehyde 2. The aldehyde is demethylated, affording phenol 3, the aldehyde group of which is reduced to benzylic alcohol 4.

A wide array of art-recognized reducing agents can be used to effect the transformation of the aldehyde 3 to the alcohol of 4. See, for example, Trost et al., COMPREHENSIVE ORGANIC SYNTHESIS: REDUCTION, Pergamon Press, 1992. In an exemplary embodiment, the reducing agent is a reagent that is a source of hydrogen which is a member selected from the group consisting of metal hydrides, and catalytic hydrogenation. In another exemplary embodiment, the reduction is an electrochemical reduction.

In another exemplary embodiment, contacting 4 with an oxidant converts it readily into the corresponding quinone 5. The oxidative conversion of 4 to 5 is optionally performed under pressure that is greater than ambient pressure. Methods for conducting reactions under pressure are recognized in the art (see, e.g., Matsumoto and Acheson, ORGANIC SYNTHESIS AT HIGH PRESSURE, J. Wiley & Sons, NY, 1991).

The hydroxyl moiety of 5 is contacted with a halogenating agent, such as thionyl chloride, affording halide 8, which can be directly coupled to a vinyl alane according to the procedure of Negishi et al., Org. Lett. 4: 261 (2002). Alternatively, the hydroxyl moiety of 5 is alkylated, giving quinone ether 7, or it is directly acylated, phosphorylated, sulfinated or sulfonated.

Rather than being oxidized to the corresponding quinone, 4 can be readily converted to a benzylic derivative with a leaving group, e.g., an oxygen-containing moiety, at the benzylic carbon. In an exemplary embodiment, the moiety is benzylic ether 6, which is prepared by contacting 4 with an alkylating agent. The benzylic ether is oxidized to quinone 7. The leaving group is replaced by coupling a reagent according to Formula (IV) and the quinone in the presence of a catalyst.

The synthetic schemes set forth herein are intended to be exemplary of the synthesis compounds of the invention. Those of skill in the art will recognize that many other synthetic strategies leading to compounds within the scope of the present invention are available. For example, by a slight modification of the starting material above, a compound having ethoxy, rather than methoxy groups is produced. Moreover, leaving and protecting groups discussed herein can be replaced with other useful groups having a similar function.

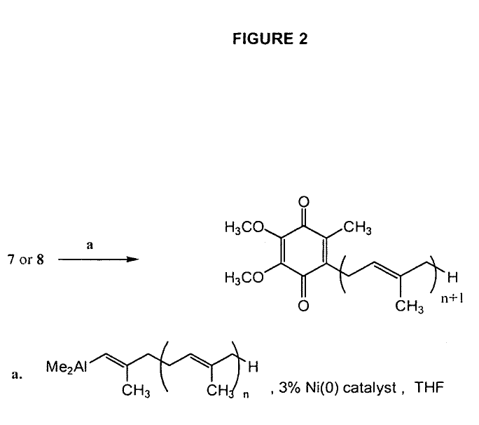

The reaction pathways set forth in FIG. 1 and FIG. 2 can be altered by using a leaving group other than a chloro at the methylene of 8. Examples of useful leaving groups are provided herein.

Moreover, the methyl group used to protect the phenol oxygen atom can be replaced with a number of other art-recognized protecting groups. Useful phenol protecting groups include, but are not limited to, ethers formed between the phenol oxygen atom and substituted or unsubstituted alkyl groups (e.g., sulfonic acid esters, methoxymethyl, benzyloxymethyl, methoxyethoxymethyl, 2-(trimethylsilyl)ethoxymethyl, methylthiomethyl, phenylthiomethyl, 2,2-dichloro-1,1-difluoroethyl, tetrahydropyranyl, phenacyl, p-bromophenacyl, cyclopropylmethyl, allyl, isopropyl, cyclohexyl, t-butyl, benzyl, 2,6-dimethylbenzyl, 4-methoxybenzyl, o-nitrobenzyl, 2,6-dichlorobenzyl, 4-(dimethylaminocarbonyl)benzyl, 9-anthrymethyl, 4-picolyl, heptafluoro-p-tolyl, tetrafluoro-4-pyridyl); silyl ethers (e.g., trimethylsilyl, t-butyldimethylsilyl); esters (e.g., acetate, levulinate, pivaloate, benzoate, 9-fluorenecarboxylate); carbonates (e.g., methyl, 2,2,2-trichloroethyl, vinyl, benzyl); phosphinates (e.g., dimethylphosphinyl, dimethylthiophosphinyl); sulfonates (e.g., methanesulfonate, toluenesulfonate, 2-formylbenzenesulfonate), and the like (see, e.g., Greene et al., PROTECTIVE GROUPS IN ORGANIC SYNTHESIS, 3rd ed., John Wiley & Sons, New York, 1999).

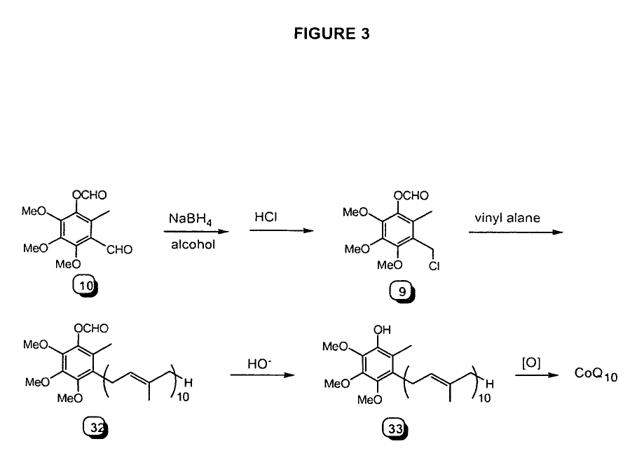

In another exemplary embodiment, the compound of the invention includes a OCH(O) moiety as the R⁶ substituent of Formula (I). As shown in FIG. 3, the OCH(O) moiety is a protecting group that remains intact during the conversion of the formyl group of 10 to the chloromethyl group of 9, and its alkylation to produce 32. The OCH(O) group is removed by hydrolytic cleavage and the resulting hydroxyl derivative 33 is readily oxidized to the corresponding ubiquinone.

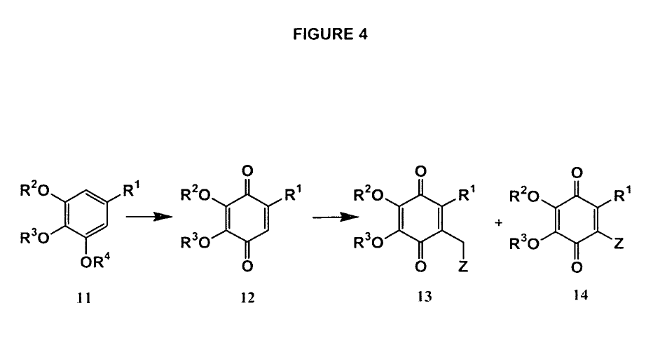

In another aspect, the invention provides a simple, inexpensive and effective purification strategy for a halomethyl quinone, prepared according to the route set forth in FIG. 4.

In the route outlined in FIG. 4, quinone 12 is prepared by oxidation of the trialkoxy (e.g., trimethoxy) starting material. The quinone is converted to the corresponding halomethyl derivative 13 by the action of formaldehyde in the presence of a selected halohydric acid. Although this route offers cost and time savings attributable to its brevity and simplicity, production of 13 gives rise to an undesired side product 14, which is difficult to remove by recrystallization or chromatography of the product mixture.

Accordingly, the invention provides a method of separating components of a mixture. The components of the mixture comprise a substituted-methylene quinone 13 and a quinone 14. R¹, R², and R³ can be independently selected from substituted or unsubstituted C₁-C₆ alkyl groups. Z is halogen, preferably chlorine. This method comprises contacting the mixture with a reactive species that selectively binds through a heteroatom to the methylene carbon of said substituted-methylene quinone, displacing said leaving group, producing a charged substituted-methylene quinone, and separating the charged substituted-methylene quinone from the quinone, thus separating the mixture.

In an exemplary embodiment, the method further comprises contacting the substituted-methylene quinone with a vinyl alane, under conditions appropriate to form a ubiquinone.

In another exemplary embodiment, the invention provides a method of separating components of a mixture. The components of the mixture comprise a substituted methylene quinone and a quinone having the formula:

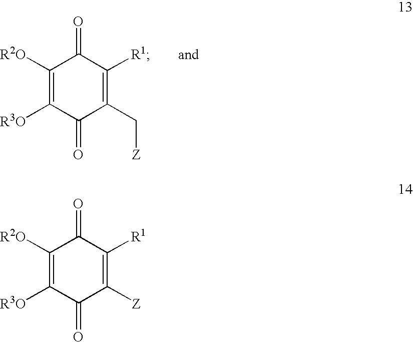

respectively. R¹, R², and R³ can be independently selected from substituted or unsubstituted C₁-C₆ alkyl groups. Z is halogen, preferably chlorine. This method comprises contacting the mixture with a reactive species that selectively binds through a heteroatom to the methylene carbon of said substituted methylene quinone and displaces the halogen. In the following step, the substituted-methylene quinone is separated from the quinone, thus separating the mixture.

In an exemplary embodiment, the reactive species is a substituted or unsubstituted C₁-C₂₀ carboxylate. In another exemplary embodiment, the separating is by chromatography. In another exemplary embodiment, the method further comprises contacting the substituted-methylene quinone with a vinyl alane, under conditions appropriate to form a ubiquinone.

In another exemplary embodiment, the invention provides an alternate route to separating a reactive substituted-methylene quinone from an analogous substituted quinone by selectively changing the halogen on the substituted-methylene quinone to a leaving group that alters the polarity of the molecule and, optionally, allows it to be crystallized away from the quinone. Thus, in one embodiment, a halogen leaving group is replaced with a charged species, e.g., (R⁹)₂S⁺ or (R⁹)₃P⁺. The marked increase in polarity of these species relative to their precursors and the quinone allow the product to be easily separated from the quinone. In exemplary cases, the charged species are solids and can be purified by crystallization.

Another method according to this embodiment relies on lowering the polarity or enhancing the hydrophobicity of the substituted-methylene quinone by converting the halogen into a species such as an ester, e.g., a carboxylate of a fatty acid, benzoic acid, etc. The increase in hydrophobic character of the desired product facilitates its separation from the quinone by recognized separation techniques, e.g., chromatography.

In another aspect, the invention provides a method of separating components of a mixture. The components of the mixture comprise a substituted-methylene quinone and a halo-quinone having the formulae:

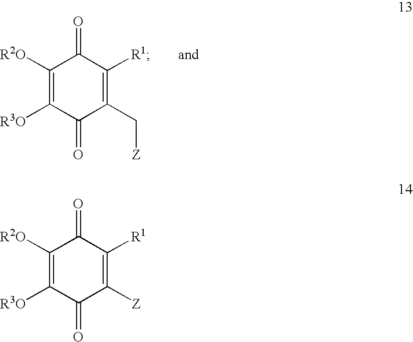

in which R¹, R², and R³ can be independently selected from substituted or unsubstituted C₁-C₆ alkyl groups. Z is a halogen. This method comprises contacting the mixture with a reducing agent that selectively reduces the halo-quinone to a halo-hydroquinone. Next, the halo-hydroquinone is contacted with a base, forming an anion of the halo-hydroquinone. Next, the anion of the halo-hydroquinone is separated from the quinone, thereby separating the mixture.

In an exemplary embodiment, the method further comprises contacting the halomethylated quinone with a vinyl alane, under conditions appropriate to form a ubiquinone. Other methods of forming ubiquinones are presented in the section entitled “Synthesis of the Products”.

In an exemplary embodiment, the mixture is contacted with a metal ion, generally used in the form of a salt or complex that preferentially reduces 14 to the corresponding hydroquinone. An exemplary metal ion is a transition metal ion, e.g., Fe(II). Basic extraction removes the acidic hydroquinone from 13.

The reducing agent, e.g., the metal ion, is present in any useful quantity. It is well within the abilities of those of skill in the art to determine both the identity, e.g., metal-containing compound, and an appropriate amount of the reducing agent for a particular purpose. For example, a vast array of data relevant to reduction and oxidation potentials of organic compounds and reducing agents, respectively, is available to those designing a purification strategy according to the instant invention.

In an exemplary embodiment, the reducing agent is a metal ion salt or complex that is sufficiently soluble in the solvent containing the desired quinone of the side product that it can be provided as a solution that is at least 0.01 mole %, preferably at least 0.05 mole %, more preferably, at least 0.1 mole %, and still more preferably, at least 0.5 mole %, in the metal ion. An exemplary species of use in the present invention is Mohr's salt, (NH₄)₂Fe(SO₄)₂. Other iron salts and metal species able to selectively transfer an electron to a haloquinone are of use in the present invention.

Alternatively, mixtures of 13 and 14 (FIG. 4) can be used directly in the coupling reaction according to the present invention. Chloromethylated quinone 13, contaminated by the corresponding chloroquinone by-product 14, can be used as a mixture of crude materials, preferably after quick filtration through a short plug of basic alumina to remove undesired components. The mixtures can for example contain up to about 50%, preferably about 0.5 to about 30% by weight of 14, which is not reacting under the appropriate conditions for the coupling.

The species purified by the strategies set forth above can then be advanced to a coupling reaction with a carboaluminated species without the need for further modification.

### Synthesis of the Carboaluminated Species

In another aspect, the invention provides a method for carboaluminating an alkyne substrate and forming a first vinyl alane. This method comprises contacting an alkyne substrate with (1) a carboalumination catalyst and (2) (L)ₚ₊₁Al in order to form the vinyl alane. The carboalumination catalyst can be any substituted and/or bridged metallocene that is capable of performing the method of the invention. In an exemplary embodiment, the carboalumination catalyst has a structure according to the following formula:

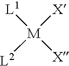

wherein M is a member selected from zirconium, titanium and hafnium. L¹ and L² are members independently selected from substituted or unsubstituted aryl, substituted or unsubstituted heteroaryl and substituted or unsubstituted cycloalkyl. L¹ and L² can optionally be covalently connected through a bridging ligand, Z. This method also has the proviso that L¹ and L² cannot both be unsubstituted cyclopentadienyl and L¹ and L² are not covalently connected to form substituted or unsubstituted tetraphenylporphyrinyl. In an exemplary embodiment, L¹ and L² are not covalently connected to form substituted or unsubstituted phenylporphyrinyl. In an exemplary embodiment, L¹ and L² are not covalently connected to form substituted or unsubstituted porphyrinyl. X′ and X″ are members independently selected from hydrogen, substituted or unsubstituted alkyl, a leaving group and an empty coordination site on M. The p in (L)ₚ₊₁Al is an integer between 1 and 2. Each L in (L)ₚ₊₁Al is a member independently selected from substituted or unsubstituted alkyl, substituted or unsubstituted alkoxy, substituted or unsubstituted aryl and substituted or unsubstituted aryloxy, thus forming said first vinyl alane. In an exemplary embodiment, each L in (L)ₚ₊₁Al is a member independently selected from substituted or unsubstituted C₁ to C₁₀ alkyl, substituted or unsubstituted C₁ to C₁₀ alkoxy, substituted or unsubstituted C₁ to C₁₀ aryl and substituted or unsubstituted C₁ to C₁₀ aryloxy.

In another exemplary embodiment, the invention provides a method of carboalumination that utilizes a metal species, e.g., a zirconium, titanium or hafnium complex, in a catalytic quantity, which means in an amount of less than 1 molar equivalent relative to the alkyne substrate. Catalysts for this reaction are referred to herein as “carboalumination catalysts”. For example, the catalyst can be present in amounts of 0.1 to 20 mole %, preferably from about 0.5 to about 5.0 mole % relative to the alkyne. It has been discovered that minimizing the amount of zirconium species present does not have a deleterious effect on the efficiency of the carboalumination. Thus, the invention provides a method of carboalumination, using a catalytic amount of a metal species, e.g., a zirconium or titanium species, that provides the carboaluminated species in high yields.

A variety of carboalumination catalysts can be used in this invention. In an exemplary embodiment, the carboalumination catalyst has a structure which is a member selected from Formula (XLIII) and Formula (XLIV):

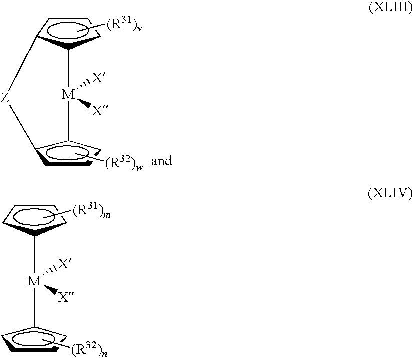

wherein M is a member selected from zirconium, titanium and hafnium. X′ and X″ are members independently selected from hydrogen and a leaving group, wherein said leaving group is a member selected from halogen, OR³⁶, OC(O)R³⁶, OS(O)R³⁶, OSO₂R³⁶, SR³⁶, S⁺(R³⁶)₂, OP(O)R³⁶R³⁷, P(O)N(R³⁶)₂(R³⁶)₂, P⁺(R³⁶)₃, P(O)R³⁸R³⁶R³⁹R³⁶. R³⁶ and R³⁷ are members independently selected from substituted or unsubstituted alkyl, substituted or unsubstituted aryl, substituted or unsubstituted heteroaryl and substituted or unsubstituted heterocycloalkyl and R³⁸ and R³⁹ are members independently selected from S and O. The indices v and w are integers independently selected from 0 to 4. When v is greater than 1, then two or more of R³¹, taken together with the carbons to which they are attached, optionally form a ring. When w is greater than 1, then two or more R³², taken together with the carbons to which they are attached, optionally form a ring. The index m is an integer selected from 0 to 5 and the index n is an integer selected from 0 to 5, with the proviso that at least one of m and n is not 0. When m is greater than 1, then two or more of R³¹, taken together with the carbons to which they are attached, optionally form a ring and when n is greater than 1, then two or more of R³², taken together with the carbons to which they are attached, optionally form a ring. R³¹ and R³² are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, OR²⁶, NR²⁶R²⁷ and —SiR²⁶R²⁷R²⁸. R²⁶, R²⁷ and R²⁸ are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl and substituted or unsubstituted heteroaryl. Z is a member selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl and —SiR^{29a}R^{29b}. R^{29a} and R^{29b} are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, —OR^{26a}, —SR^{26a}, —NR^{26a}R^{26b} and —PR^{26a}R^{26b}. R^{26a} and R^{26b} are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl and substituted or unsubstituted heteroaryl.

In an exemplary embodiment, R²⁸ and R²⁹ are independently selected from hydrogen, substituted or unsubstituted C₁ to C₁₂ alkyl, substituted or unsubstituted C₁ to C₁₂ heteroalkyl, substituted or unsubstituted C₁ to C₁₂ cycloalkyl, substituted or unsubstituted C₁ to C₁₂ heterocycloalkyl, substituted or unsubstituted C₃ to C₁₀ aryl, substituted or unsubstituted C₃ to C₁₀ heteroaryl, substituted or unsubstituted C₇ to C₁₂ arylalkyl, substituted or unsubstituted C₃ to C₁₂ heteroarylalkyl, substituted or unsubstituted C₁ to C₁₂ cycloalkyl-alkyl, and substituted or unsubstituted C₁ to C₁₂ heterocycloalkyl-alkyl. R^{26a} and R^{26b} are members independently selected from substituted or unsubstituted C₁ to C₆₀ alkyl, substituted or unsubstituted C₁ to C₆₀ cycloalkyl, substituted or unsubstituted C₁ to C₆₀ heteroalkyl, substituted or unsubstituted C₁ to C₆₀ heterocycloalkyl, substituted or unsubstituted C₃ to C₂₀ aryl and substituted or unsubstituted C₃ to C₂₀ heteroaryl. In another exemplary embodiment, Z is a member selected from substituted or unsubstituted C₁ to C₁₀ alkyl, substituted or unsubstituted C₁ to C₁₀ cycloalkyl, substituted or unsubstituted C₁ to C₁₀ heteroalkyl, substituted or unsubstituted C₁ to C₁₀ heterocycloalkyl, substituted or unsubstituted C₃ to C₂₀ aryl, substituted or unsubstituted C₃ to C₂₀ heteroaryl and —SiR^{29a}R^{29b}. In another exemplary embodiment, each R³¹ and R³² is a member independently selected from substituted or unsubstituted C₁ to C₆₀ alkyl, substituted or unsubstituted C₁ to C₆₀ cycloalkyl, substituted or unsubstituted C₁ to C₆₀ heteroalkyl, substituted or unsubstituted C₁ to C₆₀ heterocycloalkyl, substituted or unsubstituted C₃ to C₂₀ aryl, substituted or unsubstituted C₃ to C₂₀ heteroaryl, OR²⁶, NR²⁶R²⁷ and —SiR²⁶R²⁷R²⁸. R²⁶, R²⁷ and R²⁸ are members independently selected from substituted or unsubstituted C₁ to C₆₀ alkyl, substituted or unsubstituted C₁ to C₆₀ cycloalkyl, substituted or unsubstituted C₁ to C₆₀ heteroalkyl, substituted or unsubstituted C₁ to C₆₀ heterocycloalkyl, substituted or unsubstituted C₃ to C₂₀ aryl, substituted or unsubstituted C₃ to C₂₀ heteroaryl.

In an exemplary embodiment, the indices m and n are integers independently selected from 1 to 5 and R³¹ and R³² are members independently selected from substituted or unsubstituted alkyl. In another exemplary embodiment, R³¹ and R³² are members independently selected from substituted or unsubstituted C₁ to C₁₀ alkyl. Z is a member selected from —(CH₂)ᵣ—, —(CH)₂—, —Si(R²⁸R²⁹)—, —SiMe₂-, —Si(CH(Me)₂)₂—, —Si(R²⁸C(Me)₃)—, —Si(Ph)₂-, —CH₂OSi(Me₂)OCH₂—, —C(R²⁸R²⁹)ᵣ, —CH₂C(R²⁸R²⁹)CH₂— and —C(R²⁸R²⁹)ᵣ—CH₂C(R²⁸R²⁹)CH₂—, in which the index r is an integer selected from 1 to 8. R²⁸ and R²⁹ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, substituted or unsubstituted heteroarylalkyl, substituted or unsubstituted cycloalkyl-alkyl, and substituted or unsubstituted heterocycloalkyl-alkyl. In another exemplary embodiment, R²⁸ and R²⁹ are independently selected from hydrogen, substituted or unsubstituted C₁ to C₁₂ alkyl, substituted or unsubstituted C₁ to C₁₂ heteroalkyl, substituted or unsubstituted C₁ to C₁₂ cycloalkyl, substituted or unsubstituted C₁ to C₁₂ heterocycloalkyl, substituted or unsubstituted C₃ to C₁₀ aryl, substituted or unsubstituted C₃ to C₁₀ heteroaryl, substituted or unsubstituted C₇ to C₁₂ arylalkyl, substituted or unsubstituted C₃ to C₁₂ heteroarylalkyl, substituted or unsubstituted C₁ to C₁₂ cycloalkyl-alkyl, and substituted or unsubstituted C₁ to C₁₂ heterocycloalkyl-alkyl. In another exemplary embodiment, Z is a member selected from ethylene, ethenylene and dimethylsilanylene.

In another exemplary embodiment, M is zirconium. In another exemplary embodiment, X′ and X″ are leaving groups, and each leaving group is independently selected from tosylate, mesylate, brosylate, nosylate, triflate, nonaflate, tresylate, —NR^{29d}R^{29e}, endocyclic nitrogen containing heteroaryl, F, Cl, Br and I in which R^{29d} and R^{29e} are members independently selected from substituted and unsubstituted alkyl and substituted and unsubstituted heteroalkyl.

In another exemplary embodiment, the carboalumination catalyst has a structure which is a member selected from:

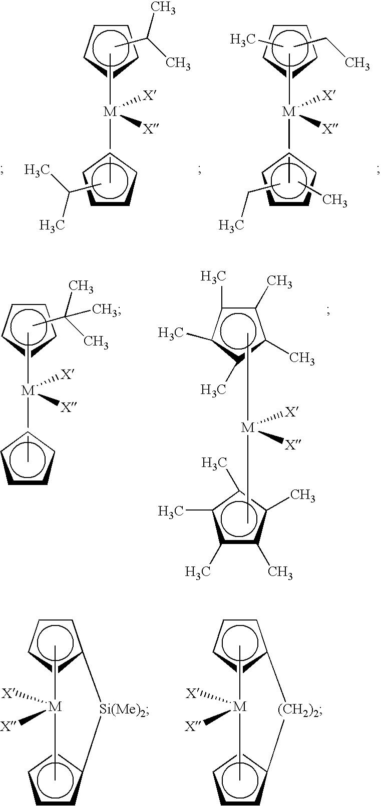

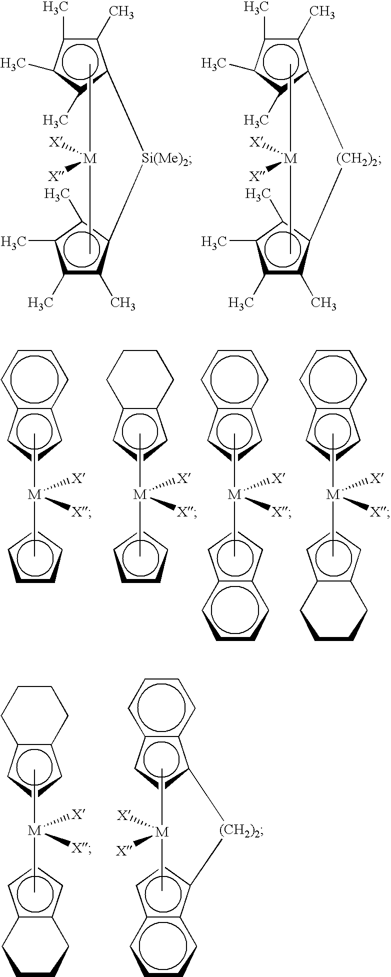

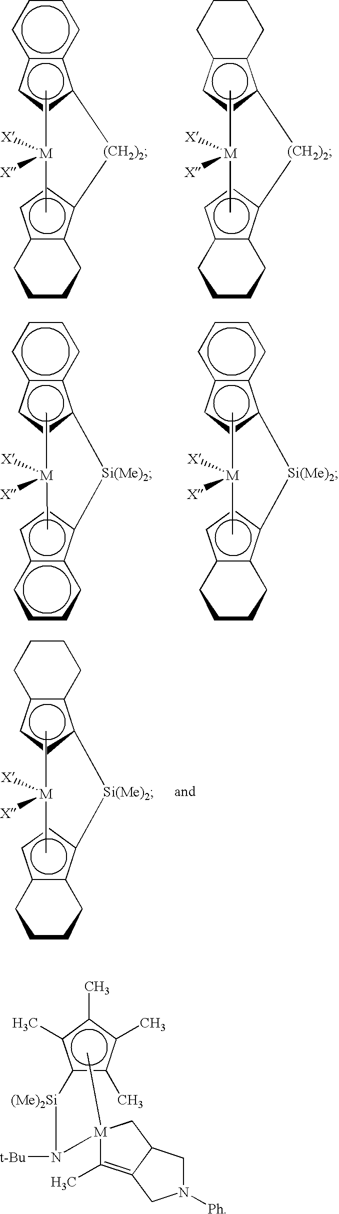

In an exemplary embodiment, wherein L¹ and L² are independently selected from substituted or unsubstituted cyclopentadienyl. In another exemplary embodiment, wherein R²³, R²⁴ and R²⁵ are methyl. In yet another exemplary embodiment, R²² is a member selected from hydrogen and —SiR²⁶R²⁷R²⁸. In still another exemplary embodiment, R²² is hydrogen.

In an exemplary embodiment, the carboalumination catalyst is a member selected from dimethyl zirconocene dimethyl; tetra-neopentyl-zirconium-(IV); tris-neopentylzirconium-(IV)-chloride; tetrakis(dimethylamino)zirconium; bis-benzyl-zirconium-(IV)-dichloride; tetra-benzyl-zirconium-(IV); tetra-chloro-bis(tetrahydrofuran) zirconium (IV); cyclopentadienylzirconium trichloride; pentamethylcyclopentadienylzirconium trichloride; (R)-biphenyl-(3,4-dimethyl-1-cyclopenta-dienyl)-zirconium (IV)—(R)-(1,1′binaphthyl-2); (S)-biphenyl-(3,4-dimethyl-1-cyclopenta-dienyl)-zirconium (IV)-(R)-(1,1′binaphthyl-2); isopropylidenebis(cyclopentadienyl) zirconium dichloride; bis-(methylcyclopentadienyl) dichloro-zirconium (IV); bis-(1,3-dimethylcyclopentadienyl)-zirconium (IV)-dichloride; bis(tetramethylcyclopentadienyl) zirconium dichloride; bis(pentamethylcyclopentadienyl) zirconium dimethyl; bis(pentamethylcyclopentadienyl)zirconium dichloride; bis(ethylcyclopentadienyl)zirconium dichloride; bis(1-ethyl-3-methylcyclopentadienyl)zirconium dichloride; bis(n-propylcyclopentadienyl) zirconium dichloride; bis(isopropylcyclopentadienyl) zirconium dichloride; isopropylidenebis (cyclopentadienyl)zirconium dichloride; bis(butylcyclopentadienyl)difluorozirconium (IV); bis(n-butylcyclopentadienyl)zirconium dichloride; bis(t-butylcyclopentadienyl)zirconium dimethyl; bis(t-butylcyclopentadienyl)zirconium dichloride; dimethylbis(t-butylcyclopentadienyl) zirconium dichloride; bis-(butylcyclopenta-dienyl)-difluoro-zirconium (IV); bis(iso-butylcyclopentadienyl)zirconium dichloride; bis(n-pentylcyclopentadienyl)zirconium dichloride; bis(n-octylcyclo-pentadienyl)zirconium dichloride; bis(n-dodecylcyclopentadienyl)zirconium dichloride; bis(trimethylsilylcyclopentadienyl)zirconium (IV) dichloride; bis[(trimethylsilyl)cyclopentadienyl]zirconium dichloride; [dimethylbis (cyclopentadienyl)silyl]zirconium dichloride; bis(cyclopentadienyl)-zirconium (IV)-(tert-butylsulfonate)-(hydride); bis(cyclopentadienyl)zirconium (IV)-(hydrido)(triflate); (cyclopentadienyl)(pentamethyl cyclopentadienyl)zirconium dichloride; (n-propylcyclopentadienyl) (tetramethylcyclopentadienyl) zirconium dichloride; (penta methylcyclopentadienyl)(n-propylcyclopentadienyl) zirconium dichloride; cyclopentadienyl(1,3-dimethylcyclopentadienyl) zirconium dichloride; dimethylsilylbis(3-n-propylcyclopentadien-1-yl)zirconium dichloride; dimethylsilylbis-(cyclopentadienyl)dichloro-zirconium-(IV); rac-dimethylsilylbis-(4-tert-butyl-2-methyl-cyclopentadienyl)-dichloro-zirconium-(IV); rac-dimethylsilylbis-(4-tert-butyl-2-methyl-cyclopentadienyl)-zirconium-(IV)-dimethyl; rac-dimethylsilylbis-(4-tert-butyl-2-methylcyclopentadienyl)dichlorozirconium (IV); rac-dimethylsilylbis-(4-tert-butyl-2-methylcyclopentadienyl)zirconium (IV) dimethyl; (cyclopentadienyl)(indenyl)zirconium dichloride; bis(indenyl)zirconium dichloride; dimethylbis(indenyl) zirconium; bis(2-methylindenyl)zirconium dichloride; dimethylbis(indenyl)zirconium dichloride; rac-dimethylsilylbis-(1-indenyl)zirconium (IV) dimethyl; rac-dimethylsilylbis(1-indenyl)zirconium dichloride; meso-dimethylsilylenebis(2-methyl-1-indenyl)zirconium (IV) dichloride; rac-dimethylsilylenebis(4,5,6,7-tetrahydro-1-indenyl)zirconium (IV) dichloride; rac-dimethylsilylbis-(4,5,6,7-tetrahydro-1-indenyl)zirconium (IV) dichloride; rac-dimethylsilylbis-(4,5,6,7-tetrahydro-1-indenyl)-zirconium-(IV)-dimethyl; rac-dimethylsilylbis-(4,5,6,7-tetrahydro-1-indenyl)-zirconium (IV)-(1,1′binaphthyl-2); dimethylsilylbis-(4,5,6,7-tetrahydro-1-indenyl)-zirconium (IV)-(R)-(1,1′binaphthyl-2); rac-ethylenebis(1-indenyl)dimethylzirconium (IV); rac-ethylenebis(indenyl)zirconium (IV) dichloride; meso-ethylenebis(1-indenyl)zirconium (IV) dichloride; rac-1,2-ethylenebis(2-methyl-1-indenyl)zirconium (IV) dichloride; rac-ethylenebis-(4,5,6,7-tetrahydro-1-indenyl)-difluorozirconium (IV); rac-ethylenebis(4,5,6,7-tetrahydro-1-indenyl) zirconium dichloride; rac-ethylenebis-(4,5,6,7-tetrahydro-1-indenyl)-difluoro-zirconium (IV); rac-ethylenebis-(4,5,6,7-tetrahydro-1-indenyl)-dimethyl-zirconium (IV); rac-ethylenebis(4,5,6,7-tetrahydro-1-indenyl)zirconium dichloride; rac-ethylenebis-(4,5,6,7-tetrahydro-1-indenyl)-zirconium (IV)-(1,1′binaphthyl-2); (R,R)-ethylenebis-(4,5,6,7-tetrahydro-1-indenyl)-dimethylzirconium (IV); (R,R)-ethylenebis-(4,5,6,7-tetrahydro-1-indenyl-indenyl)-dimethyl-zirconium (IV); (S,S)-ethylenebis-(4,5,6,7-tetrahydro-1-indenyl-dimethyl)zirconium (IV); (R,R)-ethylenebis-(4,5,6,7-tetrahydro-1-indenyl)-zirconium (IV)-(R)-(1,1′binaphthyl-2); (S,S)-ethylenebis-(4,5,6,7-tetrahydro-1-indenyl)-zirconium (IV)—(S)-(1,1′binaphthyl-2); dichloroethylenebis(indenyl)zirconium (IV); rac-dichloro-ethylenebis-(4,5,6,7-tetrahydro-1-indenyl)-zirconium (IV); dichloro-(R,R)-ethylenebis-(4,5,6,7-tetra-hydro-1-indenyl)-zirconium (IV); dichloro-(S,S)-ethylenebis-(4,5,6,7-tetra-hydro-1-indenyl)-zirconium (IV) 1,2-ethylenebis(9-fluorenyl)zirconium dichloride; [I-(9-fluorenyl)-2-(5,6-cyclopenta-2-methyl-1-indenyl)-ethane]zirconium dichloride; isopropylidene(cyclopentadienyl)(9-fluorenyl)zirconium (IV) dichloride; isopropylidene(3-methylcyclopentadienyl)(9-fluorenyl)zirconium (IV) dichloride; diphenylmethylidene(cyclopentadienyl)(2,7-di-tert-butylfluoren-9-yl)zirconium dichloride; diphenylmethylidene (cyclopentadienyl)(9-fluorenyl)zirconium dichloride; diphenylmethylene(cyclopentadienyl)(9-fluorenyl)zirconium dichloride; and [1-(9-fluorenyl)-2-(5,6-cyclopenta-2-methyl-1-indenyl)ethane]zirconium dichloride. In another exemplary embodiment, a titanium or hafnium atom is substituted for the zirconium atom in this list of carboalumination catalysts.

In another exemplary embodiment, L¹ and L² of the carboalumination catalyst primarily interact with M through pi bonding. In another exemplary embodiment, the interactions between L¹ and M, and L² and M, are between the M and carbon atoms on L¹ and L². In another exemplary embodiment, L¹ and L² are substituted or unsubstituted aryl and are covalently connected through a bridging ligand which comprises a silicon atom. In another exemplary embodiment, L¹ is substituted or unsubstituted aryl, L² is substituted or unsubstituted cycloalkyl, and L¹ and L² are covalently connected through a bridging ligand which comprises a silicon atom. In yet another exemplary embodiment, L¹ and L² are substituted or unsubstituted heteroaryl and are covalently connected through a bridging ligand which comprises a silicon atom. In still another exemplary embodiment, L¹ is substituted or unsubstituted heteroaryl, L² is substituted or unsubstituted cycloalkyl, and L¹ and L² are covalently connected through a bridging ligand which comprises a silicon atom. In still another exemplary embodiment, L¹ is substituted or unsubstituted heteroaryl, L² is substituted or unsubstituted aryl, and L¹ and L² are covalently connected through a bridging ligand which comprises a silicon atom.

In another exemplary embodiment, L¹ and L² are substituted or unsubstituted aryl and are covalently connected through a bridging ligand which comprises substituted or unsubstituted alkyl and does not comprise a silicon atom. In another exemplary embodiment, L¹ is substituted or unsubstituted aryl, L² is substituted or unsubstituted cycloalkyl, and L¹ and L² are covalently connected through a bridging ligand which comprises substituted or unsubstituted alkyl and does not comprise a silicon atom. In yet another exemplary embodiment, L¹ and L² are substituted or unsubstituted heteroaryl and are covalently connected through a bridging ligand which comprises substituted or unsubstituted alkyl and does not comprise a silicon atom. In still another exemplary embodiment, L¹ is substituted or unsubstituted heteroaryl, L² is substituted or unsubstituted cycloalkyl, and L¹ and L² are covalently connected through a bridging ligand which comprises substituted or unsubstituted alkyl and does not comprise a silicon atom. In still another exemplary embodiment, L¹ is substituted or unsubstituted heteroaryl, L² is substituted or unsubstituted aryl, and L¹ and L² are covalently connected through a bridging ligand which comprises substituted or unsubstituted alkyl and does not comprise a silicon atom.

In this embodiment, the invention is based on recognition that the remaining organometallic carboalumination catalyst (e.g., the zirconium salts), rather than the potential organic impurities, is problematic in the coupling of carboaluminated alkyne (IV) and a quinone (e.g. 13) to form a compound of Formula (III), and that minimization of the carboalumination catalyst allows for a shortened (“one pot”) route to the target ubiquinone. Thus, when a minimized amount of a zirconium or titanium species is used (e.g. ≦10 mole %), the carboaluminated product does not have to be separated prior to its being used in a coupling reaction with a quinone. Surprisingly, no marked degradation in the purity or quantity of the coupling product results from omitting the purification step.

In an exemplary embodiment, the first vinyl alane has a structure according to Formula (XL):

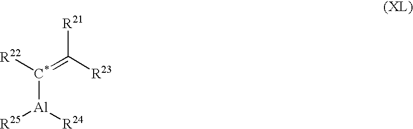

wherein R²¹ is a member selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, OR²⁶, NR²⁶R²⁷ and —SiR²⁶R²⁷R²⁸. R²² is a member selected from hydrogen, substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, OR²⁶, NR²⁶R²⁷, and —SiR²⁶R²⁷R²⁸. R²⁶, R²⁷ and R²⁸ are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl and substituted or unsubstituted heteroaryl. R²³ and R²⁴ are members independently selected from substituted or unsubstituted alkyl. R²⁵ is a member selected from a halogen and substituted or unsubstituted alkyl. The alkyne substrate has a structure according to Formula (XLI):

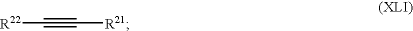

and the (L)ₚ₊₁Al has a structure according to Formula (XLII):

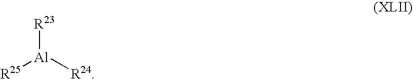

In yet another embodiment, R²¹ is a member selected from substituted or unsubstituted C₁ to C₆₀ alkyl, substituted or unsubstituted C₁ to C₆₀ cycloalkyl, substituted or unsubstituted C₁ to C₆₀ heteroalkyl, substituted or unsubstituted C₁ to C₆₀ heterocycloalkyl, substituted or unsubstituted C₃ to C₂₀ aryl, substituted or unsubstituted C₃ to C₂₀ heteroaryl, OR²⁶, NR²⁶R²⁷ and —SiR²⁶R²⁷R²⁸. R²² is a member selected from hydrogen, substituted or unsubstituted C₁ to C₆₀ alkyl, substituted or unsubstituted C₁ to C₆₀ cycloalkyl, substituted or unsubstituted C₁ to C₆₀ heteroalkyl, substituted or unsubstituted C₁-to C₆₀ heterocycloalkyl, substituted or unsubstituted C₃ to C₂₀ aryl, substituted or unsubstituted C₃ to C₂₀ heteroaryl, OR²⁶, NR²⁶R²⁷, and —SiR²⁶R²⁷R²⁸. R²⁶, R²⁷ and R²⁸ are members independently selected from substituted or unsubstituted C₁ to C₆₀ alkyl, substituted or unsubstituted C₁ to C₆₀ cycloalkyl, substituted or unsubstituted C₁ to C₆₀ heteroalkyl, substituted or unsubstituted C₁ to C₆₀ heterocycloalkyl, substituted or unsubstituted C₃ to C₂₀ aryl and substituted or unsubstituted C₃ to C₂₀ heteroaryl. R²³ and R²⁴ are members independently selected from substituted or unsubstituted C₁ to C₁₀ alkyl. R²⁵ is a member selected from a halogen and substituted or unsubstituted C₁ to C₁₀ alkyl.

In an exemplary embodiment, L¹ and L² are independently selected from substituted or unsubstituted cyclopentadiene. In another exemplary embodiment, R²³, R²⁴ and R²⁵ are methyl. In yet another exemplary embodiment, R²² is a member selected from hydrogen and —SiR²⁶R²⁷R²⁸. In yet another exemplary embodiment, R²³ is hydrogen.

In yet another exemplary embodiment, the carboaluminating occurs in a solvent which is a member selected from chlorobenzene, toluene, xylenes, mesitylene, dichloromethane, 1,2-dichloroethane, trifluoromethyltoluene, benzene and combinations thereof.

In yet another exemplary embodiment, the carboaluminating forms a second vinyl alane having a structure according to Formula (XLV):

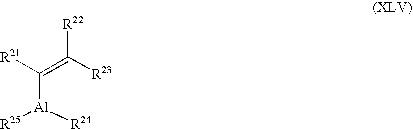

in which a ratio of the amount of first vinyl alane formed to the amount of second vinyl alane formed, is at least 90:10. In yet another exemplary embodiment, the ratio is at least 95:5.

In yet another exemplary embodiment, the alkyne substrate has a structure according to Formula (XLVI):

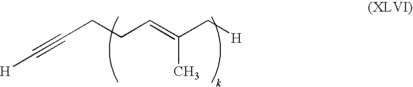

in which the index k is an integer selected from 1 to 11. The first vinyl alane has a structure according to Formula (XLVII):

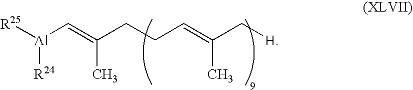

In an exemplary embodiment, the carboaluminating occurs in the presence of a member selected from water, an additive and combinations thereof. The additive is a member selected from [L³-(-Al(L³)O—)ₜ-L³] and R⁵⁰OH, in which t is an integer from 1 to 2000, L³ is substituted or unsubstituted alkyl and R⁵⁰ is a member selected from substituted or unsubstituted alkyl and substituted or unsubstituted aryl. In another exemplary embodiment, R⁵⁰ is substituted or unsubstituted C₁ to C₁₂ alkyl.

In an exemplary embodiment, the carboalumination catalyst is present in amounts of less than 10 mol-% relative to the alkyne substrate. In an exemplary embodiment, the carboalumination catalyst is present in amounts of less than 5 mol-% relative to the alkyne substrate.

In another exemplary embodiment, the alkene has a structure according to Formula (L):

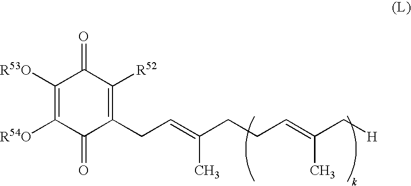

wherein R⁵², R⁵³ and R⁵⁴ are members independently selected from substituted or unsubstituted alkyl; and the index k is an integer selected from 1 to 11, and the first vinyl alane has a structure according to Formula (XLVII):

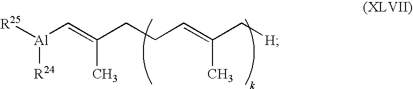

in which R²⁴ is a member selected from substituted or unsubstituted alkyl; and R²⁵ is a member selected from halogen and substituted or unsubstituted alkyl; and said compound according to Formula (XLIX) has a structure according to Formula (LI):

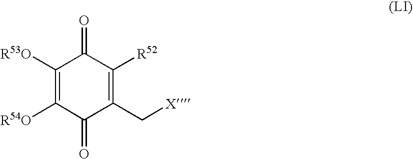

in which X′″″ is a leaving group. In an exemplary embodiment, R⁵², R⁵³ and R⁵⁴ are members independently selected from substituted or unsubstituted C₁ to C₁₀ alkyl. In another exemplary embodiment, R²⁴ is a member selected from substituted or unsubstituted C₁ to C₁₀ alkyl; and R²⁵ is a member selected from halogen and substituted or unsubstituted C₁ to C₁₀ alkyl. In an exemplary embodiment, the coupling catalyst comprises a transition metal which is a member selected from nickel, palladium, copper, iron and cobalt. In an exemplary embodiment, R⁵², R⁵³ and R⁵⁴ are each methyl and k is 9. In another exemplary embodiment, the first vinyl alane is not purified prior to the contacting of the first vinyl alane. In still another exemplary embodiment, the carboaluminating forms a second vinyl alane, and the second vinyl alane has a structure according to Formula (XLV):

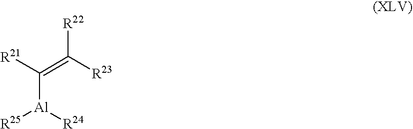

in which a ratio of the amount of the first vinyl alane formed to the amount of second vinyl alane, is at least 90:10.

In another exemplary embodiment, the invention provides a method of carboaluminating an alkyne substrate, preferably a terminal alkyne, thus forming a carboaluminated species with an alkyl moiety bound to aluminum. This method comprises contacting an alkyne substrate with a compound (L)ₚ₊₁M, wherein M is aluminum, and x equivalent of water, an alcohol R²⁰OH, or methylaluminoxane (MAO) relative to the alkyne substrate, thus carboaluminating the alkyne substrate. The symbol x can have a value between 0 to 1 (0≦x≦1). L can be a ligand independently selected from substituted or unsubstituted alkyl, alkoxy, aryl or aryloxy with 1 to 10 carbon atoms. The symbol p can be 1 or 2. In a preferred embodiment at least one of the ligands L is methyl. In a particularly preferred embodiment, (L)ₚ₊₁M is (Me)₃Al. R²⁰ is a branched or unbranched alkyl radical with 1 to 15 carbon atoms, which can be optionally substituted with 1 to 5 hydroxy substituents. Preferred alcohols R²⁰OH include methanol, ethanol, propanol, isopropanol, n-butanol, sec-butanol, tert-butanol and the like.

In an exemplary embodiment, the carboaluminated species of the method (a compound of Formula IV, for example) is utilized in a subsequent coupling reaction to a substituted methylene moiety (e.g. a compound of Formula II, for example, in which R^{5a} is CH₂OR⁷, or 13). In an exemplary embodiment, the alkyne substrate comprises a prenoidal moiety. In an exemplary embodiment, the alkyne substrate has the formula,

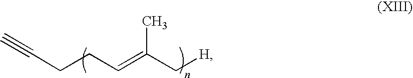

wherein n can be an integer from 0 to 19.

In another exemplary embodiment of the method for carboalumination according to the present invention, the water, the alcohol or methylaluminoxane (MAO) can be present in an amount from about 2-50 mol-% relative to said alkyne substrate.

In another exemplary embodiment, the method further comprises contacting the alkyne substrate with a carboalumination catalyst, in an amount less than one equivalent relative to the alkyne substrate. In an exemplary embodiment, the carboalumination catalyst can be a member selected from zirconium- and titanium-containing species.

In another exemplary embodiment, the carboalumination can be in a solvent mixture of a chlorinated and a non-chlorinated solvent. In another exemplary embodiment, the carboalumination can be in a non-chlorinated solvent. Suitable non-chlorinated solvents include hydrocarbons, e.g. hexanes, ligroin, toluene, petroleum ether. In a preferred embodiment, the carboalumination can be carried out in toluene or trifluoromethylbenzene or mixtures thereof.

In an exemplary embodiment, the alkyne substrate can be produced by a) forming a propyne dianion by contacting propyne with a base; and b) combining said propyne dianion with a compound according to Formula (X)

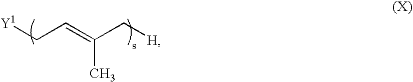

wherein Y¹ can be a leaving group, preferably halogen, e.g. chlorine, bromine or iodine, or sulfonic acid esters, e.g. tosylate or mesylate. s is an integer from 1 to 19. In an exemplary embodiment, the compound according to Formula (XII)

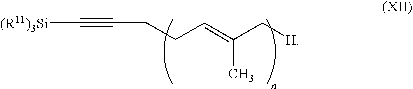

can be produced by a method comprising contacting a compound according to Formula (X) with an anion according to Formula (XI):

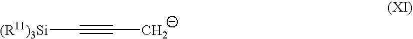

generated from (R¹¹)₃SiC≡C—CH₃ in the presence of a base.

Anion (XI) is formed in situ or, alternatively, it is formed prior to combining it with a compound according to Formula (X). The anion is formed with an appropriate base, e.g., an organolithium base.

The compound according to Formula (XII) is subsequently desilylated, e.g. using an appropriate desilylation agent such as aqueous base, alkoxides and the like, to produce a compound according to Formula (XIII):

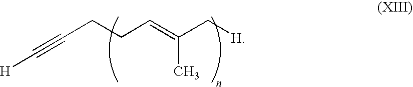

The compound of Formula (XIII) can then be carboaluminated to produce a compound according to Formula (IV).

In Formula (XI), groups represented by R¹¹ include H, substituted or unsubstituted alkyl, substituted or unsubstituted aryl, substituted or unsubstituted heteroalkyl, or a heteroatom bound to a group that satisfies the valency requirements of the heteroatom. Each R¹¹ group is selected independently of the others; they may or may not be the same as the other R¹¹ groups.

In another exemplary embodiment, the invention provides a method of carboaluminating an alkyne substrate having the formula (XIII), which comprises (a) contacting a reaction mixture comprising an alkyne substrate with an adsorbent medium; and (b) eluting the alkyne substrate from said adsorbent medium and collecting said alkyne substrate as a single fraction; and (c) submitting the product from step (b) to a carboaluminating reaction essentially without further purification, thus carboaluminating said alkyne substrate.

In an exemplary embodiment, the alkyne substrate is prepared using a derivative of solanesol and a reagent that adds a propyne synthon, e.g., a silylated-propyne in metalated form, a propargyl Grignard reagent, or a dianion of propyne. The invention also provides a quick, efficient method of purifying an alkyne, such as those produced by the methods disclosed herein. The purification method includes dissolving the crude product from the reaction in an organic solvent, e.g., petroleum ether, and passing the resulting solution through a short column of an adsorbent material, such as a chromatographic medium, e.g., silica, alumina, and the like. The so purified alkyne substrate is sufficiently pure for use in the subsequent synthetic process, e.g. the said carboalumination, without a marked degradation in yield of, or quality of product produced by, the subsequent step.

In still a further exemplary embodiment, the invention provides a method of preparing the alkyne substrate according to Formula (XIII). In this method, a propyne dianion is formed by contacting propyne with a base, e.g., n-butyllithium (n-BuLi), which is usually used in an amount of 2 to 15 equivalents. In an exemplary embodiment, the amount is of 2 to 8 equivalents, with respect to the propyne. The reaction is carried out at temperatures from −60 to 30° C. The dianion is then combined with a compound according to Formula (X).

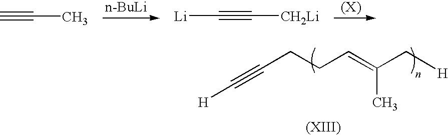

The method of the invention using propyne gas has several advantageous features. For example, propyne gas is less expensive than TMS-propyne. Moreover, use of propyne eliminates the necessity for a desilylation step, providing a two-step protocol from propyne to the solanesyl alkyne. The use of the dianion also reduces side products commonly produced from the use of the TMS-propyne mono-anion (XI).

The invention also provides an improved method for carboalumination of an alkyne substrate that utilizes both a catalytic amount of a carboalumination catalyst, e.g., a zirconium or titanium species, and a catalytic amount of water, an alcohol (R²⁰OH as defined above) or methylaluminoxane (MAO), relative to the alkyne substrate.

In an exemplary embodiment, the carboalumination method of the invention utilizes less than stoichiometric amounts of water, alcohol (R²⁰OH as defined above) or methylaluminoxane (e.g., 1-25 mole % with respect to the alkyne), in conjunction with minimization of the carboalumination (e.g., zirconocene) catalyst (e.g., 1-10 mole % with respect to the alkyne), for which no literature precedent exists. Preferably less than 1, less than 0.75, less than 0.5, 0.4, 0.3, 0.2, or 0.01 equivalents of water, alcohol or methylaluminoxane are used. Under these new conditions, the carboalumination usually proceeds to completion. Recognized methods of carboalumination utilize a stoichiometric equivalent of water relative to the alkyne substrate. See, for example, Wipf et al., Org. Lett., 2: 1713-1716 (2000) or Negishi et al., Pure Appl. Chem. 74: 151-157 (2002).

The resulting vinyl alane, the reactivity of which towards carbon electrophiles is in large measure compromised when stoichiometric amounts of water are used, retains its reactivity under these novel conditions and can be used to generate the desired product (e.g. (III)) very cleanly upon reaction with a quinone (e.g. 13) at −20° C. in high yields, usually between 70-95%.

The aluminum present in the carboaluminated species, e.g. the one of Formula (IV) can be formally neutral (an alane) or it can be charged (an aluminate). The transition metal chemistry can be catalytic or stoichiometric. For example, the alkyne substrate can be aluminated by catalytic carboalumination, forming an adduct used directly in the synthesis of a ubiquinone or, alternatively, the metalated species is transmetalated to a different reagent.

The coordination number of M is satisfied by the bonding or coordination to the metal center of the requisite number of organic ligands or substituents, such as Lewis base donors (e.g., halogen donors, oxygen donors, mercaptide ligands, nitrogen donors, phosphorus donors, and heteroaryl groups); hydrides; carbon ligands bound principally by σ-bonds (e.g., alkyls, aryls, vinyls, acyl and related ligands); carbon ligands bound by σ- and π-bonds (e.g., carbonyl complexes, thiocarbonyl, selenocarbonyl, tellurocarbonyl, carbenes, carbynes, σ-bonded acetylides, cyanide complexes, and isocyanide complexes); ligands bound through more than one atom (e.g., olefin complexes, ketone complexes, acetylene complexes, arene complexes, cyclopentadienyl complexes, π-allyl complexes); unsaturated nitrogen ligands (e.g., macrocyclic imines, dinitrogen complexes, nitric oxide complexes, diazonium complexes); and dioxygen complexes. Other useful combinations of metal ions and ligands will be apparent to those of skill in the art. See, for example, Collman et al. PRINCIPLES AND APPLICATIONS OF ORGANOTRANSITION METAL CHEMISTRY, University Science Books, 1987.

In another exemplary embodiment, the invention provides a method of carboaluminating an alkyne substrate, e.g., a terminal alkyne. The method includes contacting the alkyne substrate with a compound of the formula (L)ₚ₊₁M, wherein L, p and M are defined as above, e.g. (Me)₃Al, in an amount of 1 to 10 equivalents, preferably in an amount from 1 to 5 equivalents, especially in an amount from 1 to 2.5 equivalents, and most preferably from 1.3 to 1.8 equivalents, relative to the alkyne substrate, in the presence of less than one equivalent of water, an alcohol R²⁰OH, or alkylaluminoxane (e.g., methylaluminoxane (methyl aluminum oxide) [—Al(CH₃)O—]ₙ) relative to the alkyne substrate.

The order of addition of reactants for carrying out the method of carboalumination according to the present invention can also be varied. In an exemplary embodiment, the carboalumination catalyst and metal compound (L)ₚ₊₁M are contacted first and the alkyne substrate is subsequently added, followed by water, an alcohol (R²⁰OH) or methylaluminoxane (MAO). In an exemplary embodiment, the carboalumination catalyst and alkyne substrate are contacted first and the metal compound added subsequently, followed by the water, an alcohol (R²⁰OH) or methylaluminoxane (MAO). In an exemplary embodiment, the alkyne substrate and metal compound are contacted first and the carboalumination catalyst subsequently added, followed by water, an alcohol (R²⁰OH) or methylaluminoxane (MAO). In another exemplary embodiment, the metal compound and water, an alcohol (R²⁰OH) or methylaluminoxane (MAO) are added together and the alkyne substrate added subsequently, followed by the carboalumination catalyst.

The present invention can be conducted under a variety of conditions. For example, the carboalumination reaction can be conducted at a temperature from about −40° C. to about 50° C. In an exemplary embodiment, the temperature of the carboalumination reaction can be at about room temperature. In another exemplary embodiment, the temperature of the carboalumination reaction can be from about −20° C. to about 20° C. In another exemplary embodiment, the temperature of the carboalumination reaction can be from about −10° C. to about 12° C.

The length of time for the carboalumination reaction can vary from 30 minutes to 100 hours. In general, the lower the temperature at which the reaction is conducted, the longer the amount of time for the reaction to go to completion. For example, when the temperature is room temperature, the reaction can be completed from 9 hours to 12 hours. When the temperature is 0° C., the reaction can be completed from 19 hours to 25 hours.

The present invention also provides an unprecedented method of carboalumination utilizing solvents that are more “environmentally friendly” than art-recognized methods using halogenated solvents, e.g., dichloroethane. For example, in one embodiment, the invention provides a method of carboalumination that occurs in a solvent that includes at least one hydrocarbon (hexanes, ligroin, toluene, petroleum ether), e.g., an aromatic hydrocarbon, other than a chlorinated hydrocarbon. The solvent can be devoid of chlorinated hydrocarbons or the chlorinated solvents can be used in admixture with a solvent with less deleterious properties. Reducing or eliminating the use of halogenated solvents is a significant advance in the art.

The present method also provides an advanced approach for processing the alkyne substrate precursor to the CoQₙ₊₁ side-chain. The present method is analogous to the method of preparing the terminal alkyne set forth in U.S. Pat. No. 6,545,184. The method of the invention simplifies purification of the crude alkyne substrate (XIII) obtained, following standard workup, by filtration of the crude material through a small amount of a chromatographic medium, using an organic solvent of low polarity, e.g., petroleum ether, hexanes, etc., to elute the alkyne substrate from the medium. Importantly, the method obviates the need to fractionate the alkyne substrate, which elutes off the medium and is collected as a single fraction that contains essentially all of the small molecular organic species. An exemplary medium is a small plug of sand with an equal volume of adsorbent such as silica gel. Removal of the solvent leaves colorless to pale yellow material of ca. 70-80% purity that is ready to be used directly in the next step involving carboalumination. The purity of the material used to prepare the alkyne substrate is not critical and can be varied over a broad range of about 10-99% by weight. Material of lower purity will afford an alkyne substrate of lower purity. It was not recognized previously that use in a carboalumination of a crude alkyne substrate preparation, having only inorganics and highly polar organics removed, could provide material as pure and in as good of a yield as the use of a highly purified alkyne substrate, e.g., chromatographically purified. Alternatively, purified alkyne substrate can be used in the carboalumination.

### Synthesis of the Products

In one aspect, the method of the present invention is based on a retrosynthetic disconnection that relies on the well-known maintenance of olefin geometry in group 10 transition metal coupling reactions (Hegedus, TRANSITION METALS IN THE SYNTHESIS OF COMPLEX ORGANIC MOLECULES, University Science Books, Mill Valley, Calif., 1994). The discussion that follows focuses on a reaction, in which the coupling partners are a vinyl organometallic and a substituted-methylene quinone in which the methylene group is substituted with a leaving group (e.g., halomethyl quinone, ether, sulfonate, etc.). Please note that these reactions have similarities to coupling reactions between a vinyl alane and a protected, substituted-methylene phenol, as described in U.S. Pat. No. 6,545,184, which is herein incorporated by reference. The focus of the discussion is for clarity of illustration, and other methods and coupling partners appropriate for use in those methods will be apparent to those of skill in the art and are within the scope of the present invention.

Thus, the present invention provides a method for preparing a compound according to Formula (III):

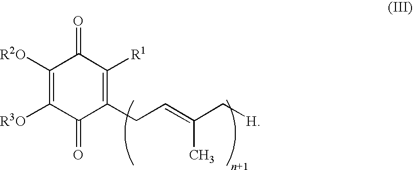

In Formula (III), each of R¹, R², R³ and n is as described above.

In one aspect, the method of the invention comprises, contacting one or more of the following substituted-methylene moieties:

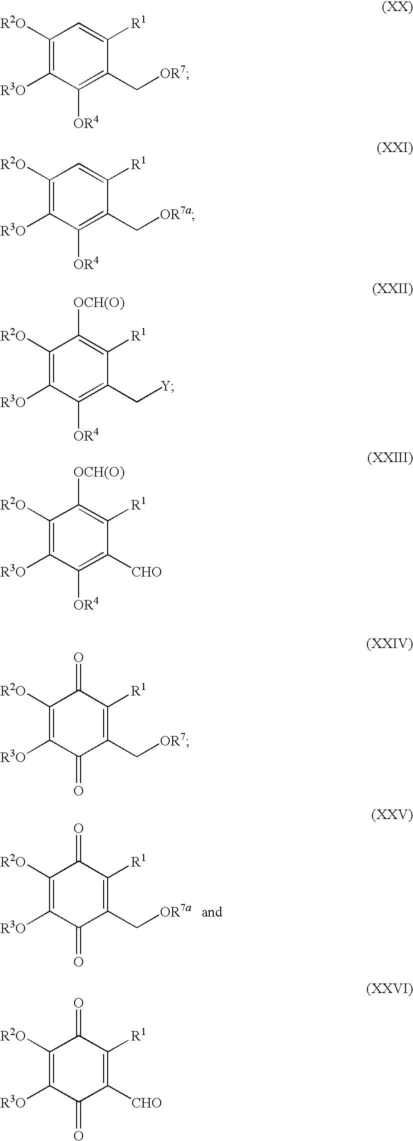

in which the substituents are as discussed above, with a carboaluminated species according to Formula (IV).

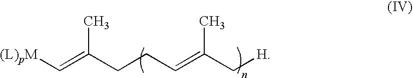

In Formula (IV), L, p, n and M are defined as above. The coupling takes place in the presence of a coupling catalyst that is effective at catalyzing coupling between the methylene carbon atom on the aromatic group or of the quinone moiety mentioned above, and the vinylic carbon attached to M on the compound according to Formula (IV).

In one embodiment of the invention compounds 7 or 8 and a compound according to Formula (IV) can be contacted in the presence of a coupling catalyst that is effective at catalyzing the coupling of the methylene carbon of a substituted methylene moiety, such as that in compounds 7 and 8, and a carboaluminated species, such as that according to Formula (IV). The coupling of compound 7 or 8 with a compound according to Formula (IV) affords the compound according to Formula (III). A representative example for preparing a ubiquinone, starting with quinone 7 or 8 (FIG. 1) is set forth in FIG. 2.

In a particularly preferred embodiment, a compound of formula 13 (e.g. compound 8) is contacted with a compound of formula (IV) derived from the carboalumination method as described above. Particular preference is given to a carboalumination process, that is conducted in the presence of substoichiometric amounts of water, an alcohol (R²⁰OH) or methylaluminoxane (MAO), and in the presence of about 0.5 to 20 mole % of a coupling catalyst (e.g. a zirconium or titanium species as described above). Preferably the subsequent coupling reaction is carried without prior removal of the carboalumination catalyst or the species derived thereof from the resulting vinyl alane. This allows one to conduct the carboalumination and the subsequent coupling as a “one pot” reaction, i.e. a reaction that is conducted in one vessel. The present methodology offers a convenient access to Coenzyme Q₁₀, which is the particularly preferred product of the methods according to this invention. The methodology offers the advantage of applicability to a technical scale.

In another exemplary embodiment, the coupling catalyst utilizes a species that comprises a transition metal. Exemplary transition metal species of use as coupling catalysts include, but are not limited to, those metals in Groups IX, X, and XI. Exemplary metals within those Groups include Cu(I), Pd(0), Co(0) and Ni(0). In an exemplary embodiment, the coupling catalyst comprises a transition metal which is a member selected from nickel, palladium, copper, iron and cobalt. Recent reports have demonstrated that catalyst couplings, using the appropriate reaction partners and based on metal catalysis, are quite general and can be used to directly afford known precursors (Naruta, J. Org. Chem., 45:4097 (1980); Eren, et al., J. Am. Chem. Soc., 110:4356 (1988) and references therein; Van Lient et al., Rec. Trav. Chim. Pays-Bays 113:153 (1994); Rüttiman et al., Helv. Chim. Acta, 73:790 (1990); Terao et al., J. Chem. Soc., Perkin Trans. 1:1101 (1978), Lipshutz et al., J. Am. Chem. Soc. 121: 11664-11673 (1999); Lipshutz et al., J. Am. Chem. Soc. 118: 5512-5313 (1999)). In another exemplary embodiment, the metal is Ni(0).

The coupling catalyst can be formed by any of a variety of methods recognized in the art. In an exemplary embodiment in which the transition metal is Ni(0), the coupling catalyst is formed by contacting a Ni(II) compound with two equivalents of a reducing agent, reducing Ni(II) to Ni(0). In an exemplary embodiment, the Ni(II) compound is NiCl₂(PPh₃)₂. In yet another exemplary embodiment, the reducing agent in n-butyllithium. In yet another exemplary embodiment, the method of the invention comprises contacting NiCl₂(PPh₃)₂, or a similar Ni species, with about two equivalents of a reducing agent (e.g., n-butyllithium), thereby reducing said NiCl₂(PPh₃)₂ to Ni(0). Alternatively, other readily available forms of Ni(0) can be employed (e.g., Ni(COD)₂).

The coupling catalyst can be a homogeneous or heterogeneous catalyst (Cornils B, Herrmann W A, APPLIED HOMOGENEOUS CATALYSIS WITH ORGANOMETALLIC COMPOUNDS: A COMPREHENSIVE HANDBOOK IN TWO VOLUMES, John Wiley and Sons, 1996; Clark J H, CATALYSIS OF ORGANIC REACTIONS BY SUPPORTED INORGANIC REAGENTS, VCH Publishers, 1994; Stiles A B, CATALYST SUPPORTS AND SUPPORTED CATALYSTS: THEORETICAL AND APPLIED CONCEPTS, Butterworth-Heinemann, 1987). In one exemplary embodiment, the coupling catalyst is supported on a solid material (e.g., charcoal, silica, etc.). In another exemplary embodiment, the coupling catalyst is a supported nickel catalyst (see, e.g., Lipshutz et al., Synthesis, 2110 (2002); Lipshutz et al., Tetrahedron 56:2139-2144 (2000); Lipshutz and Blomgren, J. Am. Chem. Soc. 121: 5819-5820 (1999); and Lipshutz et al., Inorganica Chimica Acta 296: 164-169 (1999).

The method of the invention is practiced with any useful amount of coupling catalyst effective at catalyzing coupling between the methylene carbon atom on the aromatic group or of the quinone moiety mentioned above, and the vinylic carbon attached to M on the compound according to Formula (IV). In an exemplary embodiment, the coupling catalyst is present in an amount from about 0.1 mole % to about 10 mole %. In an exemplary embodiment, the coupling catalyst is present in an amount from about 0.5 mole % to about 5 mole %. In an exemplary embodiment, the coupling catalyst is present in an amount from about 2 mole % to about 5 mole %.

The above mentioned coupling reaction can be carried out in all solvents known to those of skill in the art, suitable as solvents for transition metal catalyzed coupling reactions, e.g. ethers e.g. THF, diethyl ether and dioxane, amines e.g. triethylamine, pyridine and NMI, and others e.g. acetonitrile, acetone, ethyl acetate, DMA, DMSO, NMP and DMF. In a preferred embodiment, it is not required to completely remove the solvent in which the carboalumination was carried out, prior to the coupling.

In FIG. 2, the quinone ether 7 or the chloromethyl quinone 8 is contacted with a vinylalane in the presence of a Ni(0) catalyst. The vinyl carbon attached to M in Formula (IV) and the methylene carbon of the quinone couple, affording the corresponding ubiquinone.

The conditions of the coupling reaction can be varied. For example, the order of addition of reactants can be varied. In an exemplary embodiment, the substituted methylene moiety and carboaluminated species are contacted, and then the coupling catalyst is subsequently added. In an exemplary embodiment, the substituted methylene moiety and coupling catalyst are contacted, and then the carboaluminated species is subsequently added. In an exemplary embodiment, the coupling catalyst and carboaluminated species are contacted, and then the substituted methylene moiety is subsequently added.

The amount of the substituted methylene moiety relative to the alkyne employed in the prior carboalumination can also be varied. In an exemplary embodiment, the substituted methylene moiety, e.g. compound 8, can be reacted in amounts ranging from 0.9 to 10 equivalents relative to the alkyne mentioned above. In another exemplary embodiment, the substituted methylene moiety can be reacted in amounts ranging from 0.9 to 5 equivalents, preferably from 0.9 to 2, and most preferably from 1.1 to 1.6 equivalents, relative to the alkyne mentioned above.

The coupling reaction of the present invention can be conducted under a variety of conditions. For example, the coupling reaction can be conducted at a temperature from −40° C. to 50° C. In an exemplary embodiment, the temperature of the coupling reaction can be room temperature. In another exemplary embodiment, the temperature of the carboalumination reaction can be from −30° C. to 0° C. In another exemplary embodiment, the temperature of the carboalumination reaction can be from about −25° C. to about −15° C.

The length of time for the coupling reaction can vary from 10 minutes to 10 hours. In general, the lower the temperature at which the reaction is conducted, the longer the amount of time for the reaction to go to completion. When the temperature is about 0° C., the reaction can be completed from 30 minutes to 3 hours.

The carboalumination reaction can yield mixtures of regioisomeric vinyl alanes 26 and 26b, which in turn lead to mixtures of CoQ₁₀ (31) and its regioisomer (31b) in the subsequent coupling with the methylene carbon of chloromethylated quinone 8 as shown below. The factors influencing the regioselectivity of the carboalumination are well known to those skilled in the art. Those include for example the temperature, the nature of the solvent and of the carboalumination catalyst.

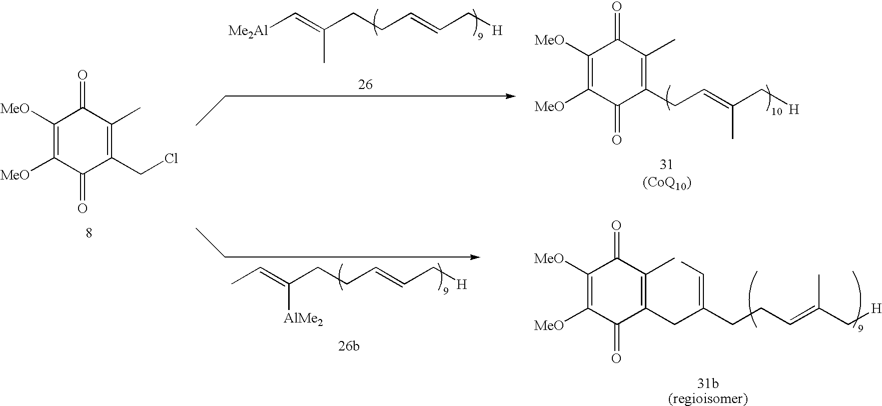

Further Processing after Coupling

The substituted methylene moiety synthesized by the method of the invention is generally oxidized to the corresponding quinone, if the moiety was not already a quinone. The phenol can be oxidized directly to the quinone or, alternatively, it can first be converted to the corresponding hydroquinone and oxidized to the quinone. An array of reagents and reaction conditions are known that oxidize phenols to quinones, see, for example, Trost B M et al. COMPREHENSIVE ORGANIC SYNTHESIS: OXIDATION, Pergamon Press, 1992.

In an exemplary embodiment, the oxidant comprises a transition metal chelate. The chelate is preferably present in the reaction mixture in an amount from about 0.1 mole % to about 10 mole %. In another exemplary embodiment, the transition metal chelate is used in conjunction with an organic base, such as an amine. Exemplary amines are the trialkyl amines, such as triethylamine. In another exemplary embodiment, the transition metal chelate is Co(salen). The chelate can be a heterogeneous or homogeneous oxidant. In an exemplary embodiment, the chelate is a supported reagent.

Alternate synthetic routes for use converting the compounds of the invention to ubiquinones, and methods to prepare useful intermediates, are provided in U.S. Pat. No. 6,545,184 to Lipshutz et al., the disclosure of which is herein incorporated by reference.

The materials, methods and devices of the present invention are further illustrated by the examples that follow. These examples are offered to illustrate, but not to limit the claimed invention.

### EXAMPLES

### General

In the examples below, unless otherwise stated, temperatures are given in degrees Celsius (° C.); operations were carried out at room or ambient temperature, “rt,” or “RT,” (typically a range of from about 18-25° C.; evaporation of solvent was carried out using a rotary evaporator under reduced pressure (typically, 4.5-30 mm Hg) with a bath temperature of up to 60° C.; the course of reactions was typically followed by thin layer chromatography (TLC) and reaction times are provided for illustration only; melting points are uncorrected; products exhibited satisfactory ¹H-NMR and/or microanalytical data; yields are provided for illustration only; and the following conventional abbreviations are also used: mp (melting point), L (liter(s)), mL (milliliters), mmol (millimoles), g (grams), mg (milligrams), min (minutes), h (hours), RBF (round bottom flask).

The following chemicals were subjected to the following preparatory steps prior to use in the Examples. PCl₃ was refluxed for 3 h at 76° C. while slowly purging with dry argon to expel HCl, distilled at atmospheric pressure and stored in a sealed container under argon until needed. DMF, 2-propanol and benzene were used as supplied from Fisher chemicals. Solanesol, purified by column chromatography on SiO₂ with 10% diethyl ether/petroleum ether, was dried azeotropically with toluene or benzene immediately prior to use. THF was distilled from Na/benzophenone ketyl prior to use. n-BuLi was obtained as a 2.5 M solution in hexanes from Aldrich and standardized by titration immediately prior to use. Ethanol was 200 proof, dehydrated, U.S.P. Punctilious grade. All other reagents were purchased from suppliers and used without further purification. Products were confirmed by ¹H NMR, ¹³C NMR, IR, LREIMS and HR-EI or HR-CI Mass Spectrometry. TLC and chromatographic solvents are abbreviated as follows: EA: ethyl acetate; PE: petroleum ether; DCM: dichloromethane.

### Example 1

### 1.1 Production of 21

### Chlorination of Solanesol

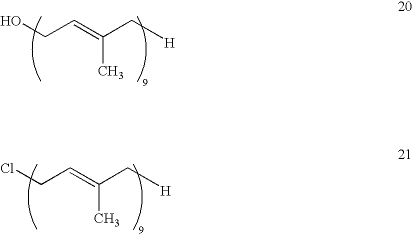

PCl₃ (180 μL, 2.10 mmol) and DMF (110 μL, 2.10 mmol) were added to a 25 mL pear-shaped flask and stirred slowly at RT for 10 min until the solution solidified into a white solid. Solanesol, 20 (2.20 g, 3.50 mmol) was dissolved in 7.0 mL THF and added via cannula to the PCl₃/DMF reagent. The heterogeneous reaction was stirred at RT for 2 h, and then the solvent was completely removed in vacuo to produce a yellow oil. Absolute ethanol (10.0 mL) was added and the flask agitated. The white precipitate was filtered to yield 2.16 g (95.1%) solanesyl chloride, 21.

### 1.2 Alternative Production of 21

### Chlorination of Solanesol

40 g (58.4 mmol) water free solanesol, 20 (purity 92% by weight) was dissolved in 158 mL (646 mmol) CCl₄ and 30.6 g (0.1168 mmol) triphenylphosphine was added at 20-25° C. The solution was heated to reflux for 6 h. After that additional 3.1 g (0.012 mmol) of triphenylphosphine was added. The solution was refluxed for 1 h and then stirred at RT for 12 h.

The resulting suspension was diluted with 125 mL n-heptane and filtered through a sintered glass filter. The resulting solution was concentrated in vacuo to remove excess CCl₄, and the resulting brown viscous residue redissolved in 125 mL n-heptane, washed 3 times with a 60:40 (v/v) mixture of methanol and water (once 62 mL, then 2 times 31 mL). A solution of brine (62 mL) was added to the combined methanolic extracts, which were extracted with heptane (62 mL). The heptane layer was separated, washed twice with a 60:40 (v/v) mixture of methanol and water (twice 32 mL). The combined heptane phases were dried over sodium sulphate, filtered and evaporated, yielding 31.2 g of a brown liquid containing 93% weight-% of solanesyl chloride, 21 (yield: 76.2%).

### 1.3 Alternative Production of 21

### Chlorination of Solanesol

23.9 g (30 mmol) of crude solanesol, 20 (purity: 79% by weight) was contacted with acetonitrile (71 mL) (biphasic mixture), 9.2 g (60 mmol) CCl₄ and 15.7 g (60 mmol) of triphenylphosphine were added. The mixture was heated to reflux for 1 h after which time TLC analysis shows complete conversion. The mixture was kept at reflux for 4 h. The reaction mixture was then extracted 3 times with n-heptane (50 mL each). The combined organic extracts were washed twice with a 60:40 (v/v) mixture of methanol and water (50 mL each) and then with brine and dried over sodium sulphate. The solvent was removed under reduced pressure to afford 22.9 g of a brown liquid containing 60.3 weight-% solanesyl chloride, 21 (yield: 71.0%).

### 1.4 Alternative Production of 21

### Chlorination of Solanesol

23.9 g (30 mmol) of crude solanesol, 20 (purity: 79% by weight) was dissolved in THF (71 mL) and 9.2 g (60 mmol) CCl₄ and 15.7 g (60 mmol) of triphenylphosphine were added. The clear solution was heated to reflux for 6 h after which time TLC analysis showed complete conversion. n-Heptane (63 mL) was added to the reaction mixture and the suspension filtered over a sintered glass frit (por. 3). The filter cake was washed with n-heptane (30 mL). The organic filtrate was washed 3 times with a 60:40 (v/v) mixture of methanol and water (30 mL each) and then with brine and dried over sodium sulphate. The solvent was removed under reduced pressure to afford 17.8 g of a brown liquid containing 80.6 weight-% solanesyl chloride, 21 (yield 73.9%).

### Example 2

### 2.1 Alkylation of Lithiated Propyne

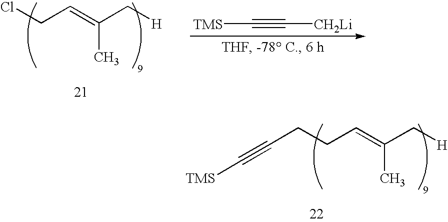

THF (4.7 mL) at −40° C. was charged with 0.36 mL n-BuLi (2.51 M in hexanes, 0.90 mmol) and after 5 min, 170 μL TMS-propyne (129 mg, 1.16 mmol) were added. After 0.75 h at −40° C., the reaction was cooled to −78° C. Crude 21 (629 mg, 0.97 mmol) dissolved in 5 mL THF was cooled to −78° C. and added slowly via cold cannula. The reaction was stirred at −78° C. for 6 h and quenched by addition of 1 mL saturated NH₄Cl solution, and the brownish-yellow mixture concentrated via rotary evaporation to a yellow oil. The residue was partitioned between 10 mL water and 10 mL petroleum ether and the layers separated. The aqueous phase was extracted 3×10 mL petroleum ether and the combined organic extracts washed with 10 mL brine, dried over anhydrous Na₂SO₄ and concentrated in vacuo. Flash chromatography (0.5% CH₂Cl₂/petroleum ether) gave the product, 22, as a clear, colorless oil which solidified upon standing (611 mg; 87%).

### 2.2 Deprotection of the Alkyne 22

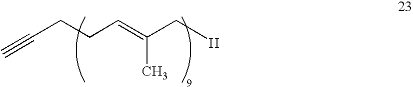

Ethanol (15 mL, 190 proof) was treated with 53 mg (2.30 mmol) freshly cut Na(0). After all solid Na(0) had dissolved, 2.76 mL of the sodium ethoxide solution (0.154 M in NaOEt, 0.43 mmol) was added to 250 mg TMS-protected alkyne substrate, 22 (0.245 mmol) A reflux condenser was attached and the reaction mixture heated to 60° C. for 4 h. Petroleum ether (10 mL) and water (10 mL) were then added, the layers separated and the aqueous layer extracted 3×10 mL petroleum ether. The combined organics were washed with 10 mL brine, dried over Na₂SO₄ and concentrated to a brown oil via rotary evaporation. Flash chromatography with 5% CH₂Cl₂/petroleum ether gave the terminal alkyne 23 (228 mg, 99%).

### 2.3 Alternative Synthesis of 23

A solution of n-butyllithium (30 mL, 75 mmol, 2.5M in hexanes, 3.75 eq) was added slowly to dry THF (60 mL), and then cooled to −7° C. Gaseous propyne (670 mL, 30 mmol, 1.5 eq) was added at −7° C. After complete addition of the propyne gas, the mixture was stirred for 1 h at −5 to 0° C., warmed to RT and stirred at that temperature for further 80 min.

A solution of solanesyl chloride, 21, (purity 75.5% by weight, 17.3 g, 20 mmol, 1.0 eq) in THF (80 mL) was then added dropwise to the aforementioned solution at temperatures between 0 and 2° C. The reaction mixture was then stirred at 0° C. for 90 min and then poured into aqueous NH₄Cl solution. The organic phase was separated, the water phase was washed once with ethyl acetate (60 mL), the combined organic phases were washed with brine, and then dried over sodium sulphate. After removal of the solvents under reduced pressure 17.6 g of a light brown oil was obtained, containing 60.0% weight-% of solanesyl alkyne substrate, 23 (yield: 80.9%).

### 2.4 Alternative Synthesis of 23

A solution of n-butyllithium (24 mL, 60 mmol, 2.5M in hexanes, 3.0 eq) was added slowly to dry THF (50 mL) at −40° C. Gaseous propyne (670 mL, 30 mmol, 1.5 eq) was gassed in at −40° C. After complete addition of the propyne gas, the cooling bath was removed and the mixture allowed to warm to 0° C., at which temperature it was stirred for additional 3 h.

A solution of solanesyl chloride, 21 (purity 92.8% by weight, 14.0 g, 20 mmol, 1.0 eq) in THF (60 mL) was then added dropwise to the aforementioned solution at temperatures between 0 and 5° C. The reaction mixture was then stirred at 0° C. for 2.5 h and then poured into aqueous NH₄Cl solution. The organic phase was separated, the water phase was washed once with ethyl acetate (50 mL), the combined organic phases were washed with brine and dried over sodium sulphate. After solvent evaporation under reduced pressure 13.8 g of a light brown oil was obtained, containing 71.8% weight-% of solanesyl alkyne, 23 (yield: 76.0%).

### 2.5 Alternative Synthesis of 23

A solution of n-butyllithium (30 mL, 75 mmol, 2.5M in hexanes, 6.25 eq) was added slowly to dry THF (60 mL) at −40° C. Propyne gas (670 mL, 30 mmol, 2.5 eq) was added to the mixture at −40° C. After complete addition of the propyne gas, the cooling bath was removed and the mixture allowed to warm to 0° C., at which temperature it is stirred for 1 h. The suspension was then warmed to RT in 30 min and stirred for 1 h at RT.

The aforementioned suspension was cooled again to −20° C.-1-25° C., and a solution of solanesyl chloride, 21 (purity 75.5% by weight, 10.24 g, 11.8 mmol, 1.0 eq) in THF (50 mL) was then added dropwise to the aforementioned solution in the same temperature interval. The reaction mixture was then stirred for 1.5 h at temperatures from −25° C. to −10° C. At −10° C., the mixture was poured into aqueous NH₄Cl solution. The organic phase was separated, the water phase was washed once with ethyl acetate (50 mL), the combined organic phases were washed with brine and dried over sodium sulphate. After solvent evaporation under reduced pressure 10.5 g of a light brown oil was obtained, containing 76.2% weight-% of solanesyl alkyne, 23 (yield: 83.0%).

### Example 3

### 3.1 Preparation of the Ni(0) catalyst 25

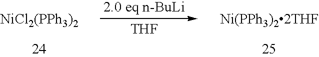

In an oven dried 5 mL round bottomed flask containing a stir bar, cooled and purged with argon, was added 24, NiCl₂(PPh₃)₂ (19.6 mg, 0.03 mmol) and the vessel was purged with argon for 2 min. THF (0.5 mL) was then added and slow stirring commenced. Slow addition of n-BuLi (0.026 mL, 0.058 mmol) gave a blood-red/black heterogeneous solution comprising 25 which was allowed to stir for 2 min prior to using it in the coupling reaction.

### Example 4

### 4.1 Oxidation of Prenoidal Phenol 30 to Quinone 31

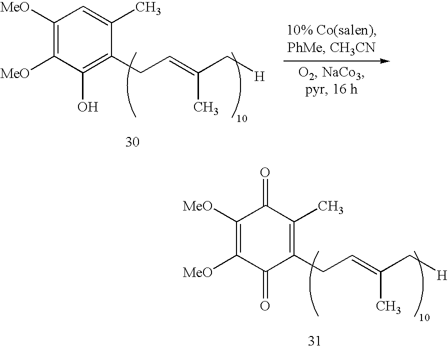

In a clean 25 mL round bottom flask and stir bar (note: not oven dried and not under argon) the phenol 30 (99.4 mg, 0.117 mmol) was dissolved in toluene (1 mL) and Na₂CO₃ (36.4 mg, 0.37 mmol) and pyridine (1 μL, 0.012 mmol) were added. Co(salen) (1.9 mg, 0.006 mmol) was then added as a red-purple solid and the reaction vessel was purged with ˜0.5 liter O₂ and held under an atmosphere of oxygen for the full reaction period. CH₃CN (150 μL) was then added to assist in solubilizing the cobalt complex. After 16 h, the reaction mixture was filtered and the supernatant was concentrated in vacuo and then chromatographed (5% EtOAc/petroleum ether) giving 68.6 mg of a red oil which solidified to a orange solid upon standing (69%). The identity of the product, 31, was confirmed by ¹H NMR, mp, HRMS and comparison to authentic sample by HPLC. Purity was established by HPLC at 98%.

### Example 5

### 5.1 Carboalumination of Alkyne 23

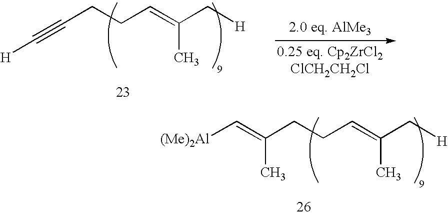

Cp₂ZrCl₂ (74 mg, 0.25 mmol) and AlMe₃ (0.5 mL, 2.0 M in hexanes, 1.0 mmol) were combined and about 90% of the solvent was removed in vacuo. The gray-white residue was then dissolved in ClCH₂CH₂Cl (DCE) (0.5 mL) giving a pale yellow solution. 23 (325 mg, 0.5 mmol) in DCE (0.25 mL) was added via cannula (exothermic) followed by washings with DCE (2×0.125 mL) to complete the transfer. After 11 h at rt, the solvent was completely removed from the heterogeneous yellow mixture in vacuo. The residue was triturated with hexanes (3×3 mL) and the hexanes removed in vacuo to remove all traces of DCE. To the heterogeneous yellow mixture was then added hexanes (2 mL) and the resulting supernatant was cannulated away from the residual Zr salts. The salts were washed twice with hexanes (2×1 mL). The washes were combined with the original wash. The combined clear yellow hexane solution containing the vinylalane 26 was then concentrated in vacuo and the residue dissolved in 0.5 mL THF (exothermic) in preparation for the cross-coupling reaction.

### 5.2 Coupling of Chloromethylated Quinone with Alane

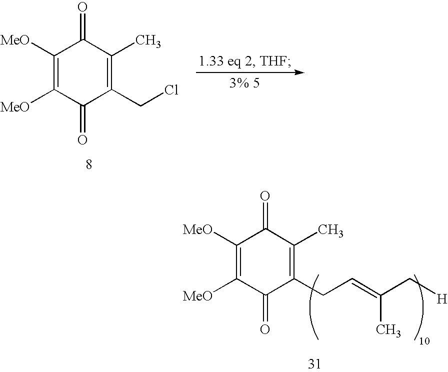

8 (86 mg, 0.375 mmol) was dissolved in THF (0.4 mL) and was cannulated into a solution of vinylalane 26. Two 0.3 mL washings of THF were used to complete the transfer of 8. The Ni(0) catalyst solution (0.188 mL, 0.011 mmol, 3 mol %) was added at RT via syringe. The solution was then protected from light and allowed to stir at RT for more than about 4 h. The reaction was quenched by the addition of EtOAc (10 mL) and 1 M HCl (20 drops). The mixture was stirred for 10 min to break up the aluminum salts (alternatively, a solution containing 0.3 g citric acid/mL water may be used to quench the reaction, followed by extraction with CHCl₃). The layers were separated and the aqueous layer was extracted with EtOAc (3×10 mL). The organics were combined, washed once with brine, dried over anhydrous Na₂SO₄ and concentrated in vacuo. The resulting yellow oil was subjected to column chromatography (10% EtOAc/petroleum ether) to give 291 mg of 31, CoQ₁₀, identical in all respects with an authentic sample.

### Example 6

### 6.1 Carboalumination of the Alkyne 23

To a flame dried, argon purged 10 mL RBF was added crude solanesol alkyne, 23 (753 mg of 73% pure material, 0.843 mmol) and Cp₂ZrCl₂ (12 mg, 0.042 mmol) and toluene (0.25 mL) at RT. The RBF was cooled to 5° C. and Me₃Al (2 M in toluene, 1.26 mmol) was added dropwise. Slight smoking was observed and the yellow mixture darkened slightly. The reaction was aged 5 min at 5° C. and then cooled to 0° C. The homogeneous mixture was aged 5 min at 0° C. and H₂O (0.75 μL, 0.042 mmol) was added. The reaction smoked slightly and immediately darkened to yellow-orange. The mixture was aged from 0 to 10° C. over 22 h (slow warming from 0° C.) after which TLC (5% DCM/PE) indicated that the alkyne was consumed. A vent needle was inserted to allow evaporation of the toluene under an argon flow, and the reaction was warmed to RT over 30 min during which time it became an orange-yellow paste containing 26. THF (1.5 mL) was added and the mixture cooled to −15° C. (slightly chunky, yellow-orange) for 10 min.

### 6.2 Coupling of the Alane 26 and Chloromethyl Quinone 8

A pre-cooled (0° C.) solution of 8 (235 mg, 1.01 mmol) in THF (0.5 mL) was added dropwise slowly to a solution containing 26 and 16.5 mg (0.025 mmol) NiCl₂(PPh₃)₂, that had been reduced by 0.050 mmol (2 equiv) n-BuLi. THF (0.5 mL) was used to assist the transfer. The reddish-orange solution was stirred at −15° C. for 3 h during which time the orange color increased. TLC (10% EA: PE) indicated a large CoQ spot, with quinone very faint. The reaction was poured into 0.25 M HCl/Et₂O and stirred 30 min. The aqueous layer extracted with Et₂O (3×10 mL) and the combined organics washed with brine, dried (anhydrous MgSO₄), filtered, concentrated in vacuo, and purified by flash chromatography (18% Et₂O: PE) to produce 31, CoQ₁₀ (550 mg, 0.639 mmol, 76% yield, orange solid). Analytical data matched that from previous experiments.

### Example 7

### 7.1 Carboalumination of an Alkyne 23

Crude solanesol alkyne, 23, was filtered over silica and the solvent (PE) evaporated. The alkyne (4.35 g of 74% pure material, 4.93 mmol, 1 eq) and Cp₂ZrCl₂ (75 mg, 0.26 mmol, 0.05 eq) were added to a flame dried, argon purged 50 mL RBF at RT. The RBF was cooled to 0° C. and Me₃Al (2 M in toluene, 3.75 mL, 7.5 mmol, 1.5 eq) was added dropwise. Slight smoking was observed and after 5-10 min a clear yellow solution was obtained. The homogeneous mixture was stirred for 30 min at 0° C. and H₂O (18 μL, 1 mmol, 0.2 eq) was added. The reaction smoked slightly and immediately darkened to yellow-orange. The mixture was stirred for 20 h at 0° C., after which time TLC (5% DCM/PE) indicated that the alkyne was consumed. The reaction was warmed to RT and the toluene was evaporated in vacuo over 50 min. The remaining orange-yellow viscous oil containing 26 was solved in THF (10 mL) and the mixture cooled to −20° C. (orange solution).

### 7.2 Coupling of the Alane 26 and Chloromethyl Quinone 8

A pre-cooled (0° C.), pre-generated Ni(0) solution (from NiCl₂ (PPh₃)_{2 {}98.1 mg, 0.15 mmol, 0.03 eq} and n-BuLi {2.5 M in hexane, 0.12 mL, 0.3 mmol, 0.06 eq} in THF {3 mL}) was added dropwise slowly at −20° C. to the previously generated solution of 26, which upon addition turned brown. To this mixture a pre-cooled (0° C.) solution of 8 (1.5 g, 92.1 wt %, 6.01 mmol, 1.2 eq) in THF (3 mL) was added dropwise slowly. The reddish-orange solution was stirred at −15° C. (±5K) for 2.5 h during which time the orange color increased. TLC (10% EA: PE) indicated a large CoQ₁₀ spot, with quinone very faint. The reaction was poured into 0.25 M HCl/Et₂O (80 mL each) and stirred for 20 min. The aqueous layer was extracted with Et₂O (2×80 mL) and the combined organics washed with brine, dried (anhydrous MgSO₄) and filtered. After removal of the solvent in vacuo 5.41 g of crude CoQ₁₀, 31 (59.2 wt %, 75.3% yield) were obtained as an orange oil.

### Example 8

### 8.1 Carboalumination of the Alkyne 23

Crude solanesol alkyne 23 was filtered over silica and the solvent (PE) evaporated. The alkyne (3.75 g of 76.5% pure material, 4.39 mmol, 1 eq) and Cp₂ZrCl₂ (75 mg, 0.26 mmol, 0.06 eq) were added to a flame dried, argon purged 50 mL RBF at RT. The RBF was cooled to 0° C. and Me₃Al (2 M in toluene, 3.75 mL, 7.5 mmol, 1.7 eq) was added dropwise. Slight smoking was observed and after 5-10 min a clear yellow solution was obtained. The homogeneous mixture was stirred for 30 min at 0° C. and H₂O (13.5 μL, 0.75 mmol, 0.17 eq) was added. The reaction smoked slightly and immediately darkened to yellow-orange. The mixture was stirred for 20 h at 0° C., after which time TLC (5% DCM/PE) indicated that the alkyne was consumed. The reaction was warmed to RT and the toluene was evaporated in vacuo over 50 min. The remaining orange-yellow viscous oil containing 26 was solved in THF (10 mL) and the mixture cooled to −20° C. (orange solution).

### 8.2 Coupling of the Alane 26 and Chloromethyl Quinone 8

A pre-cooled (0° C.), pre-generated Ni(0) solution (from NiCl₂ (PPh₃)_{2 {}98.1 mg, 0.15 mmol, 0.034 eq} and n-BuLi {2.5 M in hexane, 0.12 mL, 0.3 mmol, 0.068 eq} in THF {3 mL}) was added dropwise slowly at −20° C. to the previously prepared solution of 26, which upon addition turned brown. To this mixture a pre-cooled (0° C.) solution of 8 (1.46 g, 95 wt %, 6.01 mmol, 1.36 eq) in THF (3 mL) was added dropwise slowly. The reddish-orange solution was stirred at −15° C. (±5K) for 2.5 h during which time the orange color increased. TLC (10% EA: PE) indicated a large CoQ₁₀ spot, with quinone very faint. The reaction was poured into 0.25 M HCl/Et₂O (80 mL each) and stirred for 20 min. The aqueous layer was extracted with Et₂O (2×80 mL) and the combined organics washed with brine, dried (anhydrous MgSO₄) and filtered. After removal of the solvent in vacuo 5.05 g of crude CoQ₁₀ 31 (50.5 wt %, 67.2% yield) were obtained as an orange oil.

### Example 9

### 9.1 Carboalumination of the Alkene 23

Crude solanesol alkyne 23 was filtered over silica and the solvent (PE) evaporated. The alkyne (4.30 g of 75.9% pure material, 5.0 mmol, 1 eq) was added to a flame dried, argon purged 50 mL RBF at RT and cooled to 0° C. Me₃Al (2 M in toluene, 3.75 mL, 7.5 mmol, 1.5 eq) was added dropwise and the mixture was shaken. After 10 min a clear yellow solution was obtained, which was allowed to stir for another 25 min at 0° C. The solution was transferred to a flask containing Cp₂ZrCl₂ (75 mg, 0.26 mmol, 0.05 eq). After stirring for 30 min at 0° C. H₂O (18 μL, 1 mmol, 0.2 eq) was added. The reaction smoked slightly and immediately darkened to yellow-orange. The mixture was stirred for 20 h at 0° C., after which time TLC (5% DCM/PE) indicated that the alkyne was consumed. The reaction was warmed to RT and the toluene was evaporated in vacuo over 90 min. The remaining orange-yellow viscous oil containing 26 was solved in THF (10 mL) and the mixture cooled to −20° C. (orange solution).

### 9.2 Coupling of the Alane 26 and Chloromethyl Quinone 8

A pre-cooled (0° C.), pre-generated Ni(0) solution (from NiCl₂ (PPh₃)_{2 {}98.1 mg, 0.15 mmol, 0.03 eq} and n-BuLi {2.5 M in hexane, 0.12 mL, 0.3 mmol, 0.06 eq} in THF {3 mL}) was added dropwise slowly at −20° C. to the previously prepared solution of 26, which upon addition turned brown. To this mixture a pre-cooled (0° C.) solution of 8 (1.50 g, 92.1 wt %, 6.01 mmol, 1.2 eq) in THF (3 mL) was added dropwise slowly. The reddish-orange solution was stirred at −15° C. (±5K) for 2.5 h during which time the orange color increased. TLC (10% EA: PE) indicated a large CoQ₁₀ spot, with quinone very faint. The reaction was poured into 0.25 M HCl/EtOAc (100 mL each) and stirred for 10 min. The aqueous layer was extracted with EtOAc (2×100 mL) and the combined organics washed with brine, dried (anhydrous MgSO₄) and filtered. After removal of the solvent in vacuo 5.26 g of crude CoQ₁₀ 31 (57.0 wt %, 69.5% yield) were obtained as an orange oil.

### Example 10

### 10.1 Carboalumination of the Alkyne 23

Crude solanesol alkyne 23 was filtered over silica and the solvent (PE) evaporated. The alkyne (4.25 g of 74.1% pure material, 4.82 mmol, 1 eq) and freshly recrystallized Cp₂ZrCl₂ (75 mg, 0.26 mmol, 0.05 eq) were added to a flame dried, argon purged 50 mL RBF at RT. The RBF was cooled to 0° C. and Me₃Al (2 M in toluene, 5.0 mL, 10 mmol, 2.0 eq) was added dropwise. Slight smoking was observed and after 5-10 min a clear yellow solution was obtained. The homogeneous mixture was stirred for 30 min at 0° C. and H₂O (18 μL, 1 mmol, 0.2 eq) was added. The reaction smoked slightly and immediately darkened to yellow-orange. The mixture was stirred for 20 h at 0° C., after which time TLC (5% DCM/PE) indicated that the alkyne was consumed. The reaction was warmed to RT and the toluene was evaporated in vacuo over 90 min. The remaining orange-yellow viscous oil containing 26 was solved in THF (10 mL) and the mixture cooled to −20° C. (orange solution).

### 10.2 Coupling of the Alane 26 and Chloromethyl Quinone 8

A pre-cooled (0° C.), pre-generated Ni(0) solution (from NiCl₂ (PPh₃)_{2 {}98.1 mg, 0.15 mmol, 0.03 eq} and n-BuLi {2.5 M in hexane, 0.12 mL, 0.3 mmol, 0.06 eq} in THF {3 mL}) was added dropwise slowly at −20° C. to the previously generated solution of 26, which upon addition turned brown. To this mixture a pre-cooled (0° C.) solution of 8 (1.50 g, 92.1 wt %, 6.01 mmol, 1.2 eq) in THF (3 mL) was added dropwise slowly. The reddish-orange solution was stirred at −15° C. (±5K) for 2.5 h during which time the orange color increased. TLC (10% EA: PE) indicated a large CoQ₁₀ spot, with quinone very faint. The reaction was poured into 0.25 M HCl/EtOAc (100 mL each) and stirred for 20 min. The aqueous layer was extracted with EtOAc (2×100 mL) and the combined organics washed with brine, dried (anhydrous MgSO₄) and filtered. After removal of the solvent in vacuo 5.31 g of crude CoQ₁₀ 31 (55.6 wt %, 70.9% yield) were obtained as an orange oil.

### Example 11

### 11.1 Carboalumination of the Alkyne 23

Crude solanesol alkyne 23 was filtered over silica and the solvent (PE) evaporated. The alkyne (4.25 g of 74.1% pure material, 4.82 mmol, 1 eq) and Cp₂ZrCl₂ (75 mg, 0.26 mmol, 0.05 eq) were added to a flame dried, argon purged 50 mL RBF at RT. The RBF was cooled to 0° C. and Me₃Al (2 M in toluene, 3.0 mL, 6 mmol 1.2 eq) was added dropwise. Slight smoking was observed and after 5-10 min a clear yellow solution was obtained. The homogeneous mixture was stirred for 30 min at 0° C. and H₂O (18 μL, 1 mmol, 0.2 eq) was added. The reaction smoked slightly and immediately darkened to yellow-orange. The mixture was stirred for 20 h at 0° C., after which time TLC (5% DCM/PE) indicated that the alkyne was consumed. The reaction was warmed to RT and the toluene was evaporated in vacuo over 90 min. The remaining orange-yellow viscous oil containing 26 was solved in THF (10 mL) and the mixture cooled to −20° C. (orange solution).

### 11.2 Coupling of the Alane 26 and Chloromethyl Quinone 8

A pre-cooled (0° C.), pre-generated Ni(0) solution (from NiCl₂ (PPh₃)_{2 {}98.1 mg, 0.15 mmol, 0.03 eq} and n-BuLi {2.5 M in hexane, 0.12 mL, 0.3 mmol, 0.06 eq} in THF {3 mL}) was added dropwise slowly at −20° C. to the previously generated solution of 26, which upon addition turned brown. To this mixture a pre-cooled (0° C.) solution of 8 (1.50 g, 92.1 wt %, 6.01 mmol, 1.2 eq) in THF (3 mL) was added dropwise slowly. The reddish-orange solution was stirred at −15° C. (±5K) for 2.5 h during which time the orange color increased. TLC (10% EA: PE) indicated a large CoQ₁₀ spot, with quinone very faint. The reaction was poured into 0.25 M HCl/EtOAc (100 mL each) and stirred for 20 min. The aqueous layer was extracted with EtOAc (2×100 mL) and the combined organics washed with brine, dried (anhydrous MgSO₄) and filtered. After removal of the solvent in vacuo 5.34 g of crude CoQ₁₀ 31 (51.3 wt %, 65.9% yield) were obtained as an orange oil.

### Example 12

### 12.1 Carboalumination of the Alkyne 23

To a flame dried, argon purged 50 mL RBF was added Me₃Al (2 M in toluene, 3.75 mL, 7.5 mmol 1.5 eq). After cooling to 0° C. water (18 μL, 1 mmol, 0.2 eq) is added cautiously and stirring was continued for 30 min at 0° C. The alkyne 23 (4.30 g of 75.9% pure material, 5.0 mmol, 1 eq) was added to the yellow solution of Me₃Al and water at 0° C. After stirring for another 30 min (0° C.) the mixture was transferred to a RBF containing Cp₂ZrCl₂ (75 mg, 0.26 mmol, 0.05 eq). The resulting yellow-brown mixture was stirred for 20 h at 0° C. The reaction was warmed to RT and the toluene was evaporated in vacuo over 90 min. The remaining orange-yellow viscous oil containing 26 was solved in THF (10 mL) and the mixture cooled to −20° C. (orange solution).

### 12.2 Coupling of the Alane 26 and Chloromethyl Quinone 8

A pre-cooled (0° C.), pre-generated Ni(0) solution (from NiCl₂ (PPh₃)_{2 {}98.1 mg, 0.15 mmol, 0.03 eq} and n-BuLi {2.5 M in hexane, 0.12 mL, 0.3 mmol, 0.06 eq} in THF {3 mL}) was added dropwise slowly at −20° C. to the previously generated solution of 26, which upon addition turned brown. To this mixture a pre-cooled (0° C.) solution of 8 (1.50 g, 92.1 wt %, 6.01 mmol, 1.2 eq) in THF (3 mL) was added dropwise slowly. The reddish-orange solution was stirred at −15° C. (±5K) for 2.5 h during which time the orange color increased. TLC (10% EA: PE) indicated a large CoQ₁₀ spot, with quinone very faint. The reaction was poured into 0.25 M HCl/EtOAc (100 mL each) and stirred for 20 min. The aqueous layer was extracted with EtOAc (2×100 mL) and the combined organics washed with brine, dried (anhydrous MgSO₄) and filtered. After removal of the solvent in vacuo 5.48 g of crude CoQ₁₀ 31 (45.1 wt %, 57.2% yield) were obtained as an orange oil.

### Example 13

### 13.1 Carboalumination of the Alkyne 23

Crude solanesol alkyne 23 was filtered over silica and the solvent (PE) evaporated. The alkyne (4.21 g of 77.7% pure material, 5.0 mmol, 1 eq) and freshly recrystallized Cp₂ZrCl₂ (73.1 mg, 0.25 mmol, 0.05 eq) were added to a flame dried, argon purged 50 mL RBF at RT. The RBF was cooled to 0° C. and Me₃Al (2 M in toluene, 3.75 mL, 7.5 mmol 1.5 eq) was added dropwise. Slight smoking was observed and after 5-10 min a clear yellow solution was obtained. The homogeneous mixture was stirred for 30 min at 0° C. and H₂O (13.5 μL, 0.75 mmol, 0.15 eq) was added. The reaction smoked slightly and immediately darkened to yellow-orange. The mixture was stirred for 20 h at 0° C., after which time TLC (5% DCM/PE) indicated that the alkyne was consumed. The reaction was warmed to RT and the toluene was evaporated in vacuo over 3 h. The remaining orange-yellow viscous oil containing 26 was solved in THF (7 mL) and the mixture cooled to −20° C. (orange solution).

### 13.2 Coupling of the Alane 26 and Chloromethyl Quinone 8

A pre-cooled (0° C.), pre-generated Ni(0) solution (from NiCl₂ (PPh₃)_{2 {}98.1 mg, 0.15 mmol, 0.03 eq} and n-BuLi {2.5 M in hexane, 0.12 mL, 0.3 mmol, 0.06 eq} in THF {3 mL}) was added dropwise slowly at −20° C. to the previously prepared solution of 26, which upon addition turned brown. After aging for 5 min a pre-cooled (0° C.) solution of 8 (1.46 g, 95 wt %, 6.01 mmol, 1.36 eq) containing additional 25% dimethoxychloroquinone (DMCQ) in THF (3 mL) was added dropwise slowly. The reddish-orange solution was stirred at −15° C. (±5K) for 2 h during which time the orange color increased. TLC (10% EA: PE) indicated a large CoQ₁₀ spot, with quinone very faint. The reaction was poured into 0.25 M HCl/Et₂O (80 mL each) and stirred for 30 min. The aqueous layer was extracted with Et₂O (3×80 mL) and the combined organics washed with brine, dried (anhydrous Na₂SO₄) and filtered. After removal of the solvent in vacuo 6.07 g of crude CoQ₁₀ 31 (41.3 wt %, 58% yield) were obtained as an orange oil.

### Example 14

### 14.1 Carboalumination of the Alkyne 23

Crude solanesol alkyne 23 was filtered over silica and the solvent (PE) evaporated. The alkyne (4.00 g of 81.5% pure material, 5.0 mmol, 1 eq) and Cp₂ZrCl₂ (75 mg, 0.26 mmol, 0.05 eq) were added to a flame dried, argon purged 50 mL RBF at RT. The RBF was cooled to 0° C. and Me₃Al (2 M in toluene, 3.75 mL, 7.5 mmol 1.5 eq) was added dropwise. Slight smoking was observed and after 5-10 min a clear yellow solution was obtained. The homogeneous mixture was stirred for 30 min at 0° C. and H₂O (22.5 μL, 1.25 mmol, 0.25 eq) was added. The reaction smoked slightly and immediately darkened to yellow-orange. The mixture was stirred for 20 h at 0° C., after which time TLC (5% DCM/PE) indicated that the alkyne was consumed. The reaction was warmed to RT and the toluene was evaporated in vacuo over 90 min. The remaining orange-yellow viscous oil containing 26 was solved in THF (10 mL) and the mixture cooled to −20° C. (orange solution).

### 14.2 Coupling of the Alane 26 and Chloromethyl Quinone 8

A pre-cooled (0° C.), pre-generated Ni(0) solution (from NiCl₂ (PPh₃)_{2 {}98.1 mg, 0.15 mmol, 0.03 eq} and n-BuLi {2.5 M in hexane, 0.12 mL, 0.3 mmol, 0.06 eq} in THF {3 mL}) was added dropwise slowly at −20° C. to the previously generated solution of 26, which upon addition turned brown. To this mixture a pre-cooled (0° C.) solution of 8 (1.5 g, 92.1 wt %, 6.01 mmol, 1.2 eq) in THF (3 mL) was added dropwise slowly. The reddish-orange solution was stirred at −15° C. (±5K) for 2.5 h during which time the orange color increased. The reaction was poured into 0.25 M HCl/EtOAc (100 mL each) and stirred for 10 min. The aqueous layer was extracted with EtOAc (2×100 mL) and the combined organics washed with brine, dried (anhydrous Na₂SO₄) and filtered. After removal of the solvent in vacuo 5.25 g of crude CoQ₁₀ 31 (58.2 wt %, 70.8% yield) were obtained as an orange oil.

### Example 15

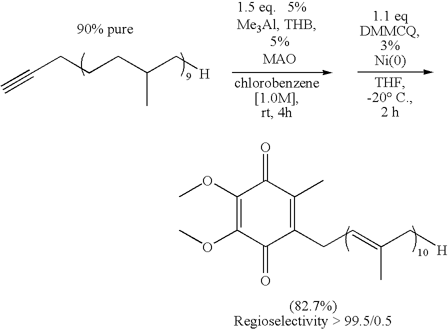

### 15.1 Carboalumination of the Alkyne 23

To a flame-dried argon purged 25 mL round bottomed flask was added solanesol alkyne 23 (221 mg @ 90% purity=199 mg pure alkyne, 0.304 mmol), ethylenebis-(tetrahydroindenyl) zirconium dichloride (THB; 6.5 mg, 0.0152 mmol, 5.0 mol %) and 0.20 mL chlorobenzene. Me₃Al (4.33 M solution in chlorobenzene, 0.10 mL, 0.456 mmol, 1.5 equiv) was added dropwise, followed by methylaluminoxane (MAO; 10% w/w solution in toluene, 10.0 μL, 0.0152 mmol, 5.0 mol %), resulting in a completely homogeneous golden orange solution containing 26. After 4 h at room temperature the solvent was completely removed under reduced pressure and replaced with 0.5 mL dry THF. The reaction was cooled to −20° C. in preparation for the coupling.

### 15.2 Coupling of the Alane 26 and Chloromethyl Quinone 8

In a 10 mL flame-dried argon purged flask 8 (77.1 mg, 0.334 mmol, 1.10 eq) was dissolved in 0.5 mL THF and transferred via cannula to the previously prepared solution of 26. In a separate 5 mL flask n-BuLi (2.5 M solution, 30 μL, 0.075 mmol) was added to a solution of NiCl₂(PPh₃)₂ (6.0 mg, 0.0091 mmol, 3.0 mol %) in 0.20 mL THF, and the active Ni(0) catalyst was immediately transferred to the previously prepared solution of 26. After 2.0 h at −20° C. the reaction was diluted with petroleum ether and quenched into 0.10M HCl_{(aq)}. The reaction was extracted with petroleum ether (3×10 mL) and dried over anhydrous Na₂SO₄. The crude material was purified by column chromatography (5% EtOAc/hexanes), isolating 217 mg (82.7%) of 31. Regioselectivity >99.5/0.5.

### Example 16

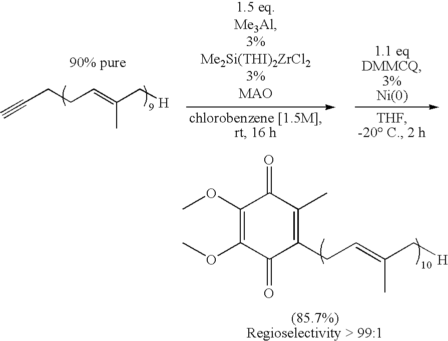

### 16.1 Carboalumination of the Alkyne 23

To a flame-dried argon purged 25 mL round bottomed flask was added column-purified solanesol alkyne 23 (457 mg @ 90% purity=411 mg pure alkyne, 0.630 mmol), dimethylsilylene-bis(tetrahydroindenyl)zirconium dichloride (8.6 mg, 0.0189 mmol, 3.0 mol %) and 0.27 mL chlorobenzene. Me₃Al (4.33 M solution in chlorobenzene, 0.22 mL, 0.945 mmol, 1.5 equiv) was added dropwise at room temperature, followed by methylaluminoxane (MAO; 10% w/w solution in toluene, 12.5 μL, 0.0189 mmol, 3.0 mol %), resulting in a completely homogeneous golden orange solution. The argon line was removed, and the flask was totally sealed off, forming a closed system. The carboalumination was given 16 h at room temperature without any disruption. At the end of 16 h, the flask was re-equipped with a vacuum/argon line, and the chlorobenzene was completely removed under reduced pressure. 1.0 mL dry THF was added to the solution containing 26, and the reaction was cooled to −20° C. in preparation for the coupling.

### 16.2 Coupling of the Alane 26 and Chloromethyl Quinone 8

In a 10 mL flame-dried argon purged flask 8 (160 mg, 0.693 mmol, 1.10 eq) was dissolved in 1.0 mL THF and transferred via cannula to the previously prepared solution of 26. In a separate 5 mL flask n-BuLi (2.5 M solution, 15 μL, 0.0378 mmol) was added to a solution of NiCl₂(PPh₃)₂ (12.4 mg, 0.0189 mmol, 3.0 mol %) in 0.5 mL THF, and the active Ni(0) catalyst was immediately transferred to the previously prepared solution of 26. After 2 h at −20° C. the reaction was diluted with petroleum ether and quenched into 0.10M HCl_{(aq)}. The aqueous phase was extracted with petroleum ether (2×20 mL) and dried over anhydrous Na₂SO₄. The crude material was purified by column chromatography (5% EtOAc/hexanes), isolating 466 mg (85.7%) of 31. Regioselectivity >99:1.

### Example 17

The ability of several carboalumination catalysts to facilitate regiospecific addition across an alkyne substrate was tested. The results of these experiments are provided in the table below. For each of experiments A-L, the following amounts of reactants were used: 0.63 mmol of alkyne substrate, 5 mol % of the carboalumination catalyst; 5 mol % of methylaluminoxane; and 0.94 mmol of Me₃Al (1.5 eq. relative to the alkyne substrate). The specific carboalumination catalyst structure, solvent, temperature, time, yield and regioselectivity data are all provided below.

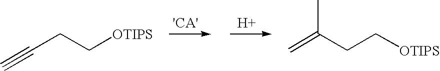

| | | | | | GC | | |
|---|---|---|---|---|---|---|---|
| Exp | Zr | Solvent | Temp | Time | Yield | Isolated | Regio |
| A | Cp₂ | DCE | rt | 2 h | 85 | | 95.7/4.3 |
| B | Cp₂ | tol | rt | 5 h | 92 | 80 | 93.6/6.7 |
| C | (i-prCp)₂ | tol | 0 | 12 h | 15 | | 100/0 |
| D | Me₂SiCp₂ | tol | 0 | 12 h | 66 | | 96.4/3.6 |
| E | t-BuCp₂ | tol | 0 | 12 h | 0 | | n/a |
| F | Cp*₂ | tol | 0 | 12 h | 0 | | n/a |
| G | THB | tol | rt | 12 h | 26 | | 100/0 |
| H | THB | CB | rt | 12 h | 52 | | 100/0 |
| I | Brintz | DCE | rt | 4 h | 98 | | 100/0 |
| J | Brintz | tol | rt | 8 h | 88 | 82 | 99.8/0.2 |
| K | Brintz | F₃CPh | rt | 8 h | 95 | | 94/6 |
| L | Brintz | CB | rt | 4 h | 89 | | 99.9/0.1 |

Cp = cyclopentadiene;
Cp* = pentamethyl cyclopentadiene;
THB = tetrahydro Brintzinger catalyst;
Brintz = Brintzinger catalyst;
CB = chlorobenzene.

### Example 18

The regiospecific addition of a halogen across an alkyne substrate was tested in this Example. As in the above example, 0.63 mmol of alkyne substrate, 5 mol % of the Brintzinger catalyst; 5 mol % of methylaluminoxane; and 0.94 mmol of Me₃Al (1.5 eq. relative to the alkyne substrate) were used. The reaction was then contacted with a bromine source (N-bromosuccinimide, or NBS) to provide the brominated product with a 74% yield.

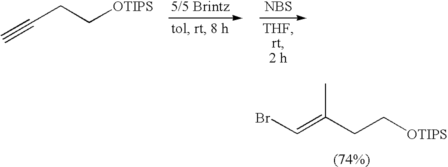

### Example 19

### E-α-Methyl-β-deuteriostyrene

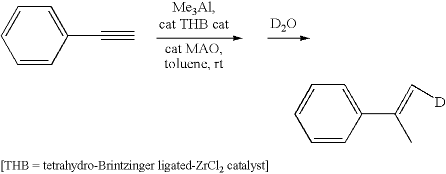

To a flame dried, argon flushed round bottom flask (25 mL) was added Brintzinger ligand (21.0 mg, 0.05 mmol) in the glove box. Trimethylaluminum in toluene (2.0 M, 0.75 mL, 1.50 mmol), methylaluminoxane (33 uL, 0.05 mmol), and the alkyne substrate (0.11 mL, 1.0 mmol) were added to the reaction flask. The mixture was allowed to stir at rt under argon. After 7.5 h, the reaction was quenched in 20 mL of deuterium oxide and petroleum ether. The crude product was then purified using flash chromatography with 5% CH₂Cl₂/pet. ether. A product yield of 84% was obtained. ¹H NMR (CDCl₃, 400 MHz) δ 7.5 (m, 2H), 7.35 (m, 2H), 7.29 (m, 1H), 5.4 (s, 1H), 2.14 (s, D), 2.10 (s, 3H); ¹³C NMR (CDCl₃, 400 MHz) δ 129.2, 128.4, 127.6, 125.7, 21.9. LREIMS (m/z, rel. %) 47(15), 51(28), 58(10), 65(7), 74(6), 78(45), 84(54), 88(6), 92(18), 104(51), 119(100), 120(11).

### Example 20

### E-2-Phenyl-4-(n-fluorophenyl)-2-butene

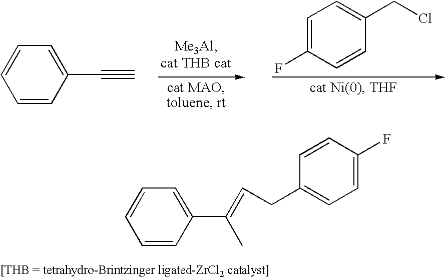

To a flame dried, argon flushed round bottom flask (25 mL) was added Brintzinger ligand (21.0 mg, 0.05 mmol) in the glove box. Trimethylaluminum in toluene (2.0 M, 0.75 mL, 1.5 mmol), methylaluminum oxane (33 uL, 0.05 mmol), and the alkyne substrate (0.11 mL, 1.0 mmol) were added to the reaction flask. The mixture was allowed to stir at rt under argon. After 5 h, toluene was removed under reduced pressure. The resulting reaction matrix was allowed to stir for 30 min under vacuum. To a flame dried, argon flushed round bottom flask (10 mL) was added NiCl₂(PPh₃)₂ (19.63 mg, 0.03 mmol) in the glove box. To another flame dried, argon flushed round bottom flask (10 mL) was added 4-fluorobenzylchloride (0.125 mL, 1.05 mmol). THF (1 mL) was then added to both flasks and the mixtures were allowed to stir until homogeneous. n-BuLi (8 μL, 0.06 mmol) was then added to the flask containing the Ni(II) solution. Both flasks were then transferred to the vinyl alane mixture via cannula. Upon complete disappearance of the vinyl alane, the reaction was then quenched with EtOAc (10 mL) followed by 1M HCl (20 drops). After extracting the aqueous layer with EtOAc (3×10 mL), the solvent was removed under reduced pressure and the crude mixture was purified using flash chromatography with 5% CH₂Cl₂/pet. ether. A product yield of 82% was obtained. ¹H NMR (CDCl₃, 400 MHz) δ 7.43 (m, 2H), 7.33 (m, 2H), 7.25 (m, 1H), 7.20 (m, 2H), 7.00 (m, 2H), 5.95 (m, J=8.0 Hz, 1H), 3.55 (d, J=7 Hz, 2H), 2.15 (m, 3H); ¹³C NMR (CDCl₃, 400 MHz) δ 129.9, 129.8, 128.4, 127.1, 126.7, 125.9, 115.5, 115.3, 34.8, 16.3; LREIMS 226, 211; HREIMS m/z calcd for C₁₆H₁₅F 226.1158. found 226.1155.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A method for carboaluminating an alkyne substrate and forming a first vinyl alane, said method comprising:
contacting said alkyne substrate with:
(1) a carboalumination catalyst, having a structure according to the following formula: 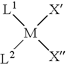
wherein
M is a member selected from zirconium, titanium and hafnium;
L¹ and L² are members independently selected from substituted or unsubstituted aryl, substituted or unsubstituted heteroaryl and substituted or unsubstituted cycloalkyl;
wherein L¹ and L² can optionally be covalently connected through a bridging ligand, Z; and
with the proviso that L¹ and L² cannot both be unsubstituted cyclopentadienyl and L¹ and L² are not covalently connected to form substituted or unsubstituted tetraphenylporphyrinyl
X′ and X″ are members independently selected from hydrogen, substituted or unsubstituted alkyl, leaving group and empty coordination site on M;
(2) (L)ₚ₊₁Al
wherein
each L is a member independently selected from substituted or unsubstituted alkyl, substituted or unsubstituted alkoxy, substituted or unsubstituted aryl and substituted or unsubstituted aryloxy; and
p is a member selected from 1 and 2;
thus forming said first vinyl alane.

2. The method according to claim 1,
wherein
said first vinyl alane has a structure according to Formula (XL): 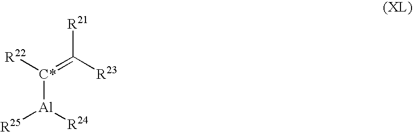
wherein
R²¹ is a member selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, OR²⁶, NR²⁶R²⁷ and —SiR²⁶R²⁷R²⁸;
R²² is a member selected from hydrogen, substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, OR²⁶, NR²⁶R²⁷ and —SiR²⁶R²⁷R²⁸
wherein
R²⁶, R²⁷ and R²⁸ are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl;
R²³ and R²⁴ are members independently selected from substituted or unsubstituted alkyl;
R²⁵ is a member selected from a halogen and substituted or unsubstituted alkyl;
said alkyne substrate has a structure according to Formula (XLI): 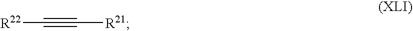
and said (L)ₚ₊₁Al has a structure according to Formula (XLII):

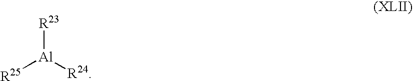

3. The method according to claim 1, wherein the carboalumination catalyst has a structure according to Formula (XLIII): 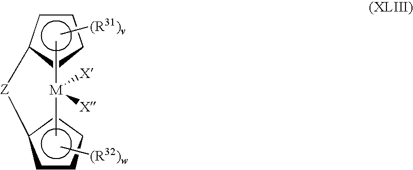
wherein
M is a member selected from zirconium, titanium and hafnium;
X′ and X″ are members independently selected from hydrogen and a leaving group, wherein said leaving group is a member selected from halogen, OR³⁶OC(O)R³⁶, OS(O)R³⁶, OSO₂R³⁶, SR³⁶, S⁺(R³⁶)₂, OP(O)R³⁶R³⁷, P(O)N(R³⁶)₂(R³⁶)₂, P⁺(R³⁶)₃, P(O)R³⁸R³⁹R³⁶
wherein
R³⁶ and R³⁷ are members independently selected from substituted or unsubstituted alkyl, substituted or unsubstituted aryl, substituted or unsubstituted heteroaryl and substituted or unsubstituted heterocycloalkyl and
R³⁸ and R³⁹ are members independently selected from S and O
v and w are integers independently selected from 0 to 4;
each R³¹ and R³² is a member independently selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, OR²⁶, NR²⁶R²⁷ and —SiR²⁶R²⁷R²⁸
wherein
R²⁶, R²⁷ and R²⁸ are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl; and
when v is greater than 1, then two or more of R³¹, taken together with the carbons to which they are attached, optionally form a ring
when w is greater than 1, then two or more R³², taken together with the carbons to which they are attached, optionally form a ring
Z is a member selected from substituted or unsubstituted alkyl, substituted or unsubstituted cycloalkyl, substituted or unsubstituted heteroalkyl, substituted or unsubstituted heterocycloalkyl, substituted or unsubstituted aryl and substituted or unsubstituted heteroaryl and —SiR^{29a}R^{29b}
wherein
R^{29a} and R^{29b} are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, —OR^{26a}, —SR^{26a}, —NR^{26a}R^{26b} and —PR^{26a}R^{26b}
wherein
R^{26a} and R^{26b} are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl.

4. The method according to claim 1, wherein the carboalumination catalyst has a structure according to Formula (XLIV): 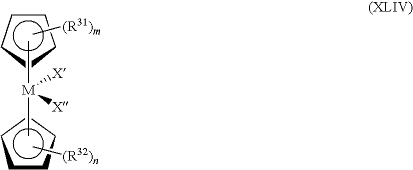
M is a member selected from zirconium, titanium and hafnium;
X′ and X″ are members independently selected from hydrogen and a leaving group, wherein said leaving group is a member selected from halogen, OR³⁶OC(O)R³⁶, OS(O)R³⁶, OSO₂R³⁶, SR³⁶, S⁺(R³⁶)₂, OP(O)R³⁶R³⁷, P(O)N(R³⁶)₂(R³⁶)₂, P⁺(R³⁶)₃, P(O)R³⁸R³⁹R³⁶
wherein
R³⁶ and R³⁷ are members independently selected from substituted or unsubstituted alkyl, substituted or unsubstituted aryl, substituted or unsubstituted heteroaryl and substituted or unsubstituted heterocycloalkyl and
R³⁸ and R³⁹ are members independently selected from S and O
m is an integer selected from 0 to 5;
n is an integer selected from 0 to 5;
with the proviso that at least one of m and n is not 0;
each R³¹ and R³² is a member independently selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, OR²⁶, NR²⁶R²⁷ and —SiR²⁶R²⁷R²⁸
wherein
R²⁶, R²⁷ and R²⁸ are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl; and
when m is greater than 1, then two or more of R³¹, taken together with the carbons to which they are attached, optionally form a ring
when n is greater than 1, then two or more of R³², taken together with the carbons to which they are attached, optionally form a ring.

5. The method according to claim 4, wherein m and n are integers independently selected from 1 to 5 and R³¹ and R³² are members independently selected from substituted or unsubstituted C₁ to C₁₀ alkyl.

6. The method according to claim 3, wherein Z is a member selected from —(CH₂)ₙ—, —(CH)₂—, —Si(R²⁸R²⁹)—, —SiMe₂-, —Si(CH(Me)₂)₂—, —Si(R²⁸C(Me)₃)—, —Si(Ph)₂-, —CH₂OSi(Me₂)OCH₂—, —C(R²⁸R²⁹)ᵣ, —CH₂C(R²⁸R²⁹)CH₂— and —C(R²⁸R²⁹)ᵣ—CH₂C(R²⁸R²⁹)CH₂—
wherein
r is an integer selected from 1 to 8;
R²⁸ and R²⁹ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted arylalkyl, substituted or unsubstituted heteroarylalkyl, substituted or unsubstituted cycloalkyl-alkyl, and substituted or unsubstituted heterocycloalkyl-alkyl.

7. The method according to claim 3, wherein Z is a member selected from ethylene, ethenylene and dimethylsilanylene.

8. The method according to claims 3 or 4, wherein M is zirconium.

9. The method according to claim 1, wherein X′ and X″ are leaving groups, and each leaving group is independently selected from tosylate, mesylate, brosylate, nosylate, triflate, nonaflate, tresylate, —NR^{29d}R^{29e}, endocyclic nitrogen containing heteroaryl, F, Cl, Br and I
wherein
R^{29d} and R^{29e} are members independently selected from substituted and unsubstituted alkyl and substituted and unsubstituted heteroalkyl.

10. The method according to claims 3 or 4, wherein said carboalumination catalyst has a structure which is a member selected from:

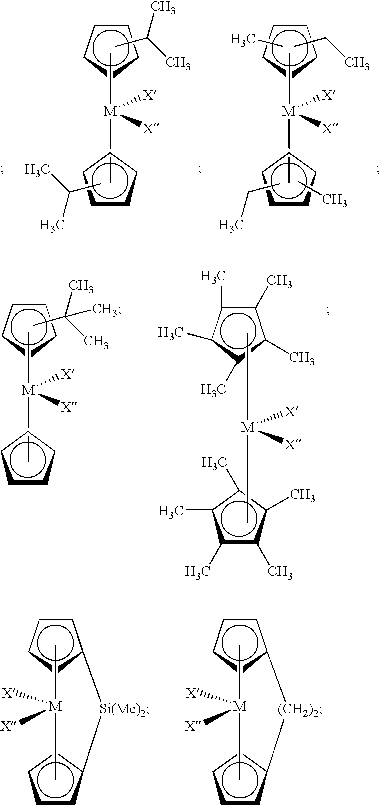

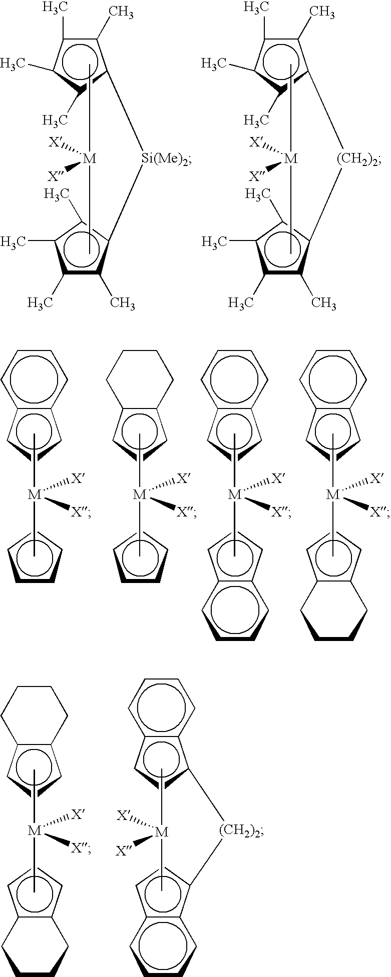

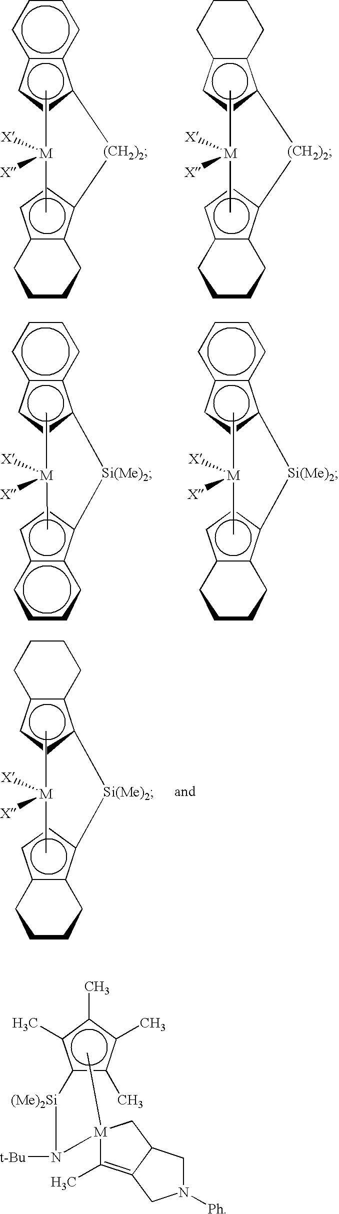

11. The method according to claim 1, wherein L¹ and L² are independently selected from substituted or unsubstituted cyclopentadienyl.

12. The method according to claim 2, wherein R²³, R²⁴ and R²⁵ are methyl.

13. The method according to claim 2, wherein R²² is a member selected from hydrogen and —SiR²⁶R²⁷R²⁸.

14. The method according to claim 13, wherein R²² is hydrogen.

15. The method according to claim 1, wherein said carboaluminating occurs in a solvent which is a member selected from chlorobenzene, toluene, xylenes, mesitylene, dichloromethane, 1,2-dichloroethane, trifluoromethyltoluene, benzene and combinations thereof.

16. The method according to claim 2, wherein the carboaluminating forms a second vinyl alane having a structure according to Formula (XLV): 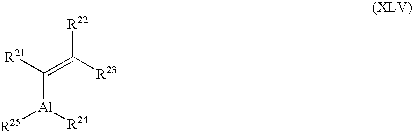
wherein
a ratio of the amount of first vinyl alane formed to the amount of second vinyl alane formed, is at least 90:10.

17. The method according to claim 16, wherein said ratio is at least 95:5.

18. The method according to claim 2, wherein said alkyne substrate has a structure according to Formula (XLVI): 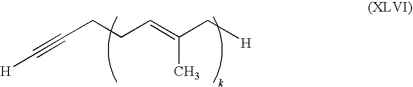
wherein
k is an integer selected from 1 to 11; and
said first vinyl alane has a structure according to Formula (XLVII):

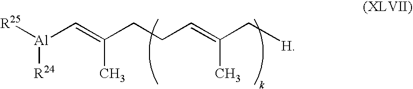

19. The method according to claim 1, wherein the carboaluminating occurs in the presence of a member selected from water, an additive and combinations thereof.

20. The method according to claim 19, wherein the additive is a member selected from [L³-(-Al(L³)O—)ₜ-L³] and R⁵⁰OH
wherein
t is an integer selected from 1 to 2000;
L³ is substituted or unsubstituted alkyl and
R⁵⁰ is a member selected from substituted or unsubstituted alkyl and substituted or unsubstituted aryl.

21. The method according to claims 3 or 4, wherein said carboalumination catalyst is present in amounts of less than 10 mol-% relative to the alkyne substrate.

22. The method according to claim 21, wherein said carboalumination catalyst is present in amounts of less than 5 mol-% relative to the alkyne substrate.

23. A method for the preparation of an alkene having a structure according to Formula (XLVIII): 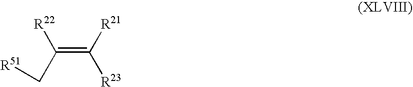
wherein
R²¹ is member selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl;
R²² is a member selected from hydrogen, substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, OR²⁶, NR²⁶R²⁷ and —SiR²⁶R²⁷R²⁸
R²³ is a member selected from substituted or unsubstituted alkyl;
R⁵¹ is a member selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl,
said method comprising:
contacting said first vinyl alane according to claim 2 with a compound having a structure according to Formula (XLIX) 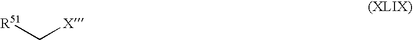
wherein
X′″ is a leaving group,
in the presence of a coupling catalyst effective at catalyzing coupling between the methylene carbon in Formula (XLIX) and C* in Formula (XL),
thereby preparing said alkene.

24. The method according to claim 23, wherein said alkene has a structure according to Formula (L): 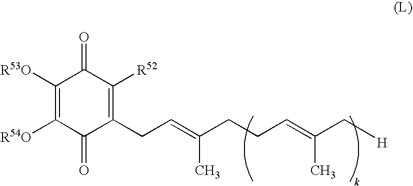
wherein
R⁵², R⁵³ and R⁵⁴ are members independently selected from substituted or unsubstituted alkyl; and
k is an integer selected from 1 to 11;
said first vinyl alane has a structure according to Formula (XLVII): 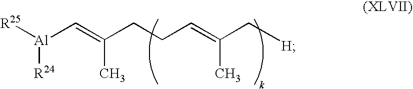
wherein
R²⁴ is a member selected from substituted or unsubstituted alkyl; and
R²⁵ is a member selected from halogen and substituted or unsubstituted alkyl;
and said compound according to Formula (XLIX) has a structure according to Formula (L1): 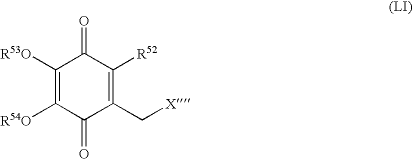
wherein
X′″ is a leaving group.

25. The method according to claim 23, wherein the coupling catalyst comprises a transition metal which is a member selected from nickel, palladium, copper, iron and cobalt.

26. The method according to claim 23, wherein the first vinyl alane is not purified prior to said contacting.

27. The method according to claim 24, wherein R⁵², R⁵³ and R⁵⁴ are each methyl and k is 9.

28. The method according to claim 24, wherein the carboaluminating forms a second vinyl alane, and the second vinyl alane has a structure according to Formula (XLV): 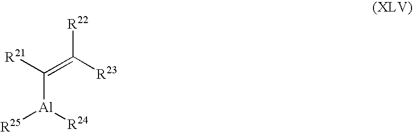
wherein
a ratio of the amount of first vinyl alane formed to the amount of second vinyl alane, is at least 90:10.

29. A method of using a carboalumination catalyst to form a vinyl alane, said method comprising contacting said carboalumination catalyst with an alkyne substrate, thereby forming said vinyl alane
wherein said carboalumination catalyst has a structure according to Formula (XLIII): 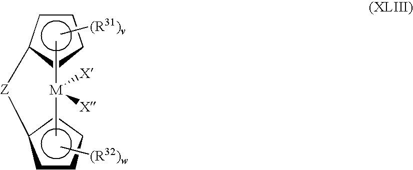
wherein
M is a member selected from zirconium, titanium and hafnium;
X′ and X″ are members independently selected from hydrogen and a leaving group, wherein said leaving group is a member selected from halogen, OR³⁶OC(O)R³⁶, OS(O)R³⁶, OSO₂R³⁶, SR³⁶, S⁺(R³⁶)₂, OP(O)R³⁶R³⁷, P(O)N(R³⁶)₂(R³⁶)₂, P⁺(R³⁶)₃, P(O)R³⁸R³⁶R³⁶
wherein
R³⁶ and R³⁷ are members independently selected from substituted or unsubstituted alkyl, substituted or unsubstituted aryl, substituted or unsubstituted heteroaryl and substituted or unsubstituted heterocycloalkyl and
R³⁸ and R³⁹ are members independently selected from S and O
v and w are integers independently selected from 0 to 4;
each R³¹ and R³² is a member independently selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, OR²⁶, NR²⁶R²⁷ and —SiR²⁶R²⁷R²⁸
wherein
R²⁶, R²⁷ and R²⁸ are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl; and
when v is greater than 1, then two or more of R³¹, taken together with the carbons to which they are attached, optionally form a ring
when w is greater than 1, then two or more R³², taken together with the carbons to which they are attached, optionally form a ring
Z is a member selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl and —SiR^{29a}R^{29b}
wherein
R^{29a} and R^{29b} are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, —OR^{26a}, —SR^{26a}, —NR^{26a}R^{26b} and —PR^{26a}R^{26b}
wherein
R^{26a} and R^{26b} are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl and substituted or unsubstituted heteroaryl.

30. A method of using a carboalumination catalyst to form a vinyl alane, said method comprising contacting said carboalumination catalyst with an alkyne substrate, thereby forming said vinyl alane
wherein said carboalumination catalyst has a structure according to Formula (XLIV): 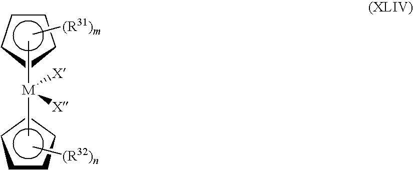
M is a member selected from zirconium, titanium and hafnium;
X′ and X″ are members independently selected from hydrogen and a leaving group, wherein said leaving group is a member selected from halogen, OR³⁶, OC(O)R³⁶, OS(O)R³⁶, OSO₂R³⁶, SR³⁶, S⁺(R³⁶)₂, OP(O)R³⁶R³⁷, P(O)N(R³⁶)₂(R³⁶)₂, P⁺(R³⁶)₃, P(O)R³⁸R³⁹R³⁶
wherein
R³⁶ and R³⁷ are members independently selected from substituted or unsubstituted alkyl, substituted or unsubstituted aryl, substituted or unsubstituted heteroaryl and substituted or unsubstituted heterocycloalkyl and
R³⁸ and R³⁹ are members independently selected from S and O
m is an integer selected from 0 to 5;
n is an integer selected from 0 to 5;
with the proviso that at least one of m and n is not 0;
R³¹ and R³² are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl, substituted or unsubstituted heteroaryl, OR²⁶, NR²⁶R²⁷ and —SiR²⁶R²⁷R²⁸
wherein
R²⁶, R²⁷ and R²⁸ are members independently selected from substituted or unsubstituted alkyl, substituted or unsubstituted cycloalkyl, substituted or unsubstituted heteroalkyl, substituted or unsubstituted heterocycloalkyl, substituted or unsubstituted aryl and substituted or unsubstituted heteroaryl; and
when m is greater than 1, then two or more of R³¹, taken together with the carbons to which they are attached, optionally form a ring; and
when n is greater than 1, then two or more of R³², taken together with the carbons to which they are attached, optionally form a ring.
